(19) United States
(12) Patent Application Publication
(10) Pub. No.: US 2026/0327362 A1
(43) Pub. Date: Oct. 22, 2026

(54) ANTIPATHOGENIC POLYMERS

(71) Applicant: The Boeing Company, Arlington, VA (US)

(72) Inventor: Michael MONTEIRO, Brisbane (AU)

(73) Assignee: The Boeing Company, Arlington, VA (US)

(21) Appl. No.: 19/485,613

(22) PCT Filed: May 15, 2024
(86) PCT No.: PCT/US2024/029421

§ 371 Date: Nov. 18, 2025

Related U.S. Application Data
(60) Provisional application No. 63/467,775, filed on May 19, 2023.

(51) Int. Cl. C08F 220/56; C09D 5/02; C09D 7/20; C09D 133/26

(52) U.S. Cl. CPC C08F 220/56; C09D 5/022; C09D 7/20; C09D 133/26

(57) ABSTRACT

The present disclosure provides a method and composition of disposing a polymer on a surface. The method includes depositing the polymer on a surface. The polymer includes a plurality of N-alkylacrylamide units. The compound includes a moiety represented by the formula: wherein R¹ is C₁-C₂₀ alkyl. The compound includes a moiety represented by the formula: wherein R², R³, and R⁴ are each independently hydrogen or C₁-C₂₀ alkyl. The compound includes at least two moieties represented by the formula: wherein Q is O or N, R** is C₁-C₂₀ alkyl, R⁶, R⁷, and R⁸ are independently C₁-C₆ alkyl or hydrogen and R⁹, if present, is C₁-C₁₆ alkyl, C₁-C₆ alkylyne, azole, guanidine, an oligomer of guanidine such as diguanidine, polysaccharide, chromophore, and combination(s) thereof, q is an integer of 0 or 1. At least one q is 1.

### RELATED APPLICATIONS

This application is a U.S. national phase entry of PCT/US2024/029421 filed on May 15, 2024, and published as WO 2024/242956 on Nov. 28, 2024, and claims benefit of and priority to U.S. Provisional Patent Application No. 63/467,775, filed May 19, 2023, the entire contents of all of which are incorporated herein by reference.

### FIELD

The present disclosure provides an antipathogenic polymer, such as a coating of antipathogenic polymers over a surface of a vehicle, a building, a wearable, a filter, or any other suitable object.

### BACKGROUND

Pandemics have major and lingering impacts on society. Reduction in pathogen transmission can be achieved on/in high touch surfaces and enclosed environments including vehicles, offices, transportation facilities, habitation, among others by preventing the transmission of pathogens, such as viruses and microbes.

One goal of airlines includes aircraft cabin sterility and space transportation, and habitation industries are concerned with preventing the transmission of pathogens, such as viruses and microbes. Indeed, certain pathogens, e.g., mRNA pathogens such as SARS-CoV-2 and variants thereof, are sense RNA viruses. Sense RNA viruses are capable of protein translation even after lysis of the host cell occurs. This feature of such mRNA pathogens makes it challenging to prevent pathogen transmission because standard cell lysing techniques are often ineffective against these sense RNA viruses.

Currently disease transmission reduction techniques focus on improving mechanical features of air filtration systems. Unfortunately, this does not reduce or stop the transmission of pathogens via surfaces. Other disease prevention techniques focus on lysing the host cell having the pathogen using a polymer or emulsion. However, these techniques can fail to deactivate RNA and RNA translation of sense RNA viruses due to poor innervation with the virus. These techniques often require non-functional groups, such as styrene, to act as a backbone for the functional groups, which can agglomerate, reducing the available surface area of the polymer or emulsion for the RNA virus to bind to the functional groups. As such, sense RNA viruses may linger for hours or days on surfaces.

Therefore, there is a need for antipathogenic surface coatings that do not agglomerate and are effective at both reducing the transmission of pathogens and deactivating pathogen genetic material. Moreover, these antipathogenic surface coatings should be simple, safe, and easily scalable for production.

### SUMMARY

The present disclosure provides a method of disposing a polymer. The method includes depositing the polymer on a surface. The polymer includes a plurality of N-alkylacrylamide units. The compound includes a moiety represented by the formula:

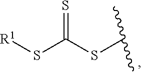

wherein R¹ is C₁-C₂₀ alkyl. The compound includes a moiety represented by the formula:

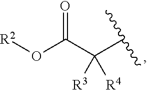

wherein R², R³, and R⁴ are each independently hydrogen or C₁-C₂₀ alkyl. The compound includes at least two moieties represented by the formula:

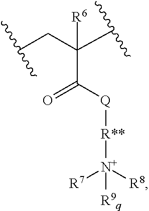

wherein Q is O or N, R** is C₁-C₂₀ alkyl, R⁶, R⁷, and R⁸ are independently C₁-C₆ alkyl or hydrogen and R⁹, if present, is C₁-C₁₆ alkyl, C₁-C₆ alkylyne, azole, guanidine, an oligomer of guanidine such as diguanidine, polysaccharide, chromophore, such as coumarin, and combination(s) thereof. q is an integer of 0 or 1. At least one q is 1.

The present disclosure also provides a polymer. The polymer includes a plurality of N-isopropylacrylamide units. The compound includes a moiety represented by the formula:

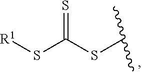

wherein R¹ is C₁-C₂₀ alkyl. The compound includes a moiety represented by the formula:

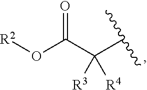

wherein R², R³, and R⁴ are each independently hydrogen or C₁-C₂₀ alkyl. The polymer includes at least two moieties represented by the formula:

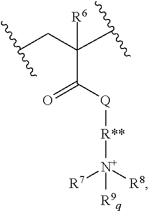

wherein Q is O or N, R** is C₁-C₂₀ alkyl, R⁶, R⁷, and R⁸ are independently C₁-C₆ alkyl or hydrogen and R⁹, if present, is C₁-C₁₆ alkyl, C₁-C₆ alkylyne, azole, guanidine, an oligomer of guanidine such as diguanidine, polysaccharide, chromophore, and combination(s) thereof. q is an integer of 0 or 1. At least one q is 1.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only typical aspects of this present disclosure and are therefore not to be considered limiting of its scope, for the present disclosure may admit to other equally effective aspects.

FIG. 1 depicts a ¹H NMR (in CDCl₃) spectrum of a crude polymer, according to certain aspects of the present disclosure.

FIG. 2 depicts a ¹H NMR (in DMSO-d6) of purified polymer, according to certain aspects of the present disclosure.

FIG. 3 depicts a ¹H NMR (in DMSO-d6) spectrum of a crude polymer, according to certain aspects of the present disclosure.

FIG. 4 depicts a ¹H NMR (in DMSO-d6) spectrum of a purified polymer, according to certain aspects of the present disclosure.

FIG. 5 depicts SEC traces of a first polymer “A” and a second polymer “B”. (DMAc as the eluent for GPC, RI signals used to plot), according to certain aspects of the present disclosure.

FIG. 6 is a schematic illustration of functionalization of P(DMAEMA-co-NIPAM), according to certain aspects of the present disclosure.

FIG. 7 depicts a synthetic process of quaternization of a polymer with diguanidine chloroacetamide (digua-Cl) at 60° C., according to certain aspects of the present disclosure.

FIG. 8 depicts ¹H NMR (in DMSO-d6) spectra of the mixture before and after reactions, with highlighted region showing full consumption of the starting material digua-Cl, according to certain aspects of the present disclosure.

FIGS. 9A and 9B depict ¹H NMR (in DMSO-d6) spectra, according to certain aspects of the present disclosure. FIG. 9A depicts a ¹H NMR (in DMSO-d6) spectrum of the purified sample after reaction with digua-Cl. FIG. 9B depicts a ¹H NMR (in DMSO-d6) spectrum of the unfunctionalized polymer.

FIG. 10 depicts a synthetic process of quaternization of a polymer with propargyl bromide at 60° C., according to certain aspects of the present disclosure.

FIGS. 11A and 11B depict ¹H NMR (in DMSO-d6) spectra, according to certain aspects of the present disclosure. FIG. 11A depicts a ¹H NMR (in DMSO-d6) spectra of the purified sample after reaction with propargyl bromide. FIG. 11B depicts a ¹H NMR (in DMSO-d6) spectra of the unfunctionalized polymer.

FIG. 12 depicts a synthetic process of quaternization of a polymer with 1-iodooctane at 60° C., according to certain aspects of the present disclosure.

FIGS. 13A and 13B depict ¹H NMR (in DMSO-d6) spectra, according to certain aspects of the present disclosure. FIG. 13A depicts a ¹H NMR (in DMSO-d6) spectra of the purified sample after reaction with 1-iodooctane. FIG. 13B depicts a ¹H NMR (in DMSO-d6) spectra of the unfunctionalized polymer.

FIG. 14 depicts a synthetic process of copper catalyzed azide alkyne cycloaddition (CuAAC) coupling with polysugar azide and coumarin azide, according to certain aspects of the present disclosure.

FIG. 15 depicts a ¹H NMR (in DMSO-d6) spectrum of the purified sample after CuAAC reaction, according to certain aspects of the present disclosure.

FIG. 16 depicts an alternate synthetic process of quaternization of a polymer with propargyl bromide at 60° C., according to certain aspects of the present disclosure.

FIG. 17 depicts an alternate synthetic process of quaternization of a polymer with propargyl bromide at 60° C., according to certain aspects of the present disclosure.

FIGS. 18A and 18B depict ¹H NMR (in DMSO-d6) spectra, according to certain aspects of the present disclosure. FIG. 18A depicts a ¹H NMR (in DMSO-d6) spectra of the purified sample after reaction with propargyl bromide. FIG. 18B depicts a ¹H NMR (in DMSO-d6) spectra of the unfunctionalized polymer.

FIGS. 19A and 19B depict ¹H NMR (in DMSO-d6) spectra, according to certain aspects of the present disclosure. FIG. 19A depicts a ¹H NMR (in DMSO-d6) spectra of the purified sample after reaction with 1-iodooctane. FIG. 19B depicts a ¹H NMR (in DMSO-d6) spectra of the unfunctionalized polymer.

FIG. 20 depicts an alternate synthetic process of copper catalyzed azide alkyne cycloaddition (CuAAC) coupling with polysugar azide and coumarin azide, according to certain aspects of the present disclosure.

FIG. 21 depicts a ¹H NMR (in DMSO-d6) spectrum of the purified sample after CuAAC reaction, according to certain aspects of the present disclosure.

FIG. 22 depicts a fluorescent test of functionalized polymers, according to certain aspects of the present disclosure.

FIG. 23 depicts a synthesis route of functionalized polymers based on RAFT polymerization, according to certain aspects of the present disclosure.

FIG. 24 depicts a ¹H NMR (400 MHz, CDCL₃, 298K) spectra of MacroCTA-B after purification, according to certain aspects of the present disclosure.

FIG. 25 depicts an SEC trace of MacroCTA-B using a RI detector (eluent DMAC) with PSTY standards, according to certain aspects of the present disclosure.

FIG. 26 depicts a synthesis route of quaternization of MacroCTA-B: P(NIPAM₄₇-co-DMAEMA₃₀), according to certain aspects of the present disclosure.

FIG. 27 depicts ¹H NMR (in DMSO-d₆) spectra of purified samples after quaternization with iodooctane, propargyl bromide, and diguanidine, according to certain aspects of the present disclosure.

FIG. 28 depicts a SEC trace of quaternized MacroCTA-B, according to certain aspects of the present disclosure.

FIG. 29 depicts a synthetic CuAAC ‘click’ route of q-CTA-B with P(galactose)-N₃ and 3-azido-7-hydroxycoumarin, according to certain aspects of the present disclosure.

FIGS. 30A and 30B depict ¹H NMR (in D₂O) spectra of purified samples, according to certain aspects of the present disclosure. FIG. 30A depicts a ¹H NMR (in D₂O) spectrum of purified samples before click with P(galactose)-N₃, 3-azido-7-hydroxycoumarin. FIG. 30B depicts a ¹H NMR (in D₂O) spectrum of purified samples after click with P(galactose)-N₃, 3-azido-7-hydroxycoumarin.

FIGS. 31A and 31B depict fluorescence of functionalized polymer due to attachment of coumarin groups (10 mg/mL in H₂O), according to certain aspects of the present disclosure. FIG. 31A depicts fluorescence of functionalized polymer before click with P(galactose)-N₃, 3-azido-7-hydroxycoumarin. FIG. 31B depicts fluorescence of functionalized polymer after click with P(galactose)-N₃, 3-azido-7-hydroxycoumarin.

FIG. 32 depicts a synthetic route of functional polymer based on free radical polymerization, according to certain aspects of the present disclosure.

FIG. 33 depicts a ¹H NMR spectrum (400 MHz, CDCl₃, 298K) of P(NIPAM₅₀-co-DMAEMA₃₀) after purification, according to certain aspects of the present disclosure.

FIG. 34 depicts a SEC trace of P(NIPAM₅₀-co-DMAEMA₃₀), according to certain aspects of the present disclosure.

FIG. 35 depicts a synthetic route of a sequential quaternization of P(NIPAM₅₀-co-DMAEMA₃₀), according to certain aspects of the present disclosure.

FIG. 36 depicts ¹H NMR (in DMSO-d₆) spectra of purified samples after quaternization with iodooctane, propargyl bromide, and diguanidine, according to certain aspects of the present disclosure.

FIG. 37 depicts a synthetic CuAAC ‘click’ route of q-P(NIPAM₅₀-co-DMAEMA₃₀) with P(galactose)-N₃ and 3-azido-7-hydroxycoumarin, according to certain aspects of the present disclosure.

FIGS. 38A and 38B depict ¹H NMR (in D₂O) spectra of purified samples of functionalized P(NIPAM₅₀-co-DMAEMA₃₀), according to certain aspects of the present disclosure. FIG. 38A depicts a ¹H NMR (in D₂O) spectrum of purified samples of P(NIPAM₅₀-co-DMAEMA₃₀) before click with P(galactose)-N₃, 3-azido-7-hydroxycoumarin. FIG. 38B depicts a ¹H NMR (in D₂O) spectrum of purified samples of P(NIPAM₅₀-co-DMAEMA₃₀) after click with P(galactose)-N₃, 3-azido-7-hydroxycoumarin.

FIGS. 39A and 39B depict fluorescence of functionalized P(NIPAM₅₀-co-DMAEMA₃₀) due to attachment of coumarin groups (10 mg/mL in H₂O), according to certain aspects of the present disclosure. FIG. 39A depicts fluorescence of functionalized P(NIPAM₅₀-co-DMAEMA₃₀) before click with P(galactose)-N₃, 3-azido-7-hydroxycoumarin. FIG. 39B depicts fluorescence of functionalized P(NIPAM₅₀-co-DMAEMA₃₀) after click with P(galactose)-N₃, 3-azido-7-hydroxycoumarin.

### DETAILED DESCRIPTION

The rise in coronavirus variants and other sense RNA viruses or DNA viruses has resulted in surges of the disease across the globe. For example, the mutations in the spike protein on the surface of the virion membrane not only allow for greater transmission but raise concerns about vaccine effectiveness. Preventing the spread of SARS-CoV-2, variants of SARS-CoV-2 and other sense RNA viruses from person-to-person via airborne or surface transmission requires effective inactivation of the virus.

The present disclosure provides a polymer, which may be a coating including an antipathogenic polymer that may be used to treat a surface of a vehicle, a building, a wearable, a filter, or any other suitable object having a porous or non-porous composition, such as a solid and/or a woven or non-woven fabric. The coating has antipathogenic properties that are effective at reducing or eliminating pathogens and/or reducing the transmission of pathogens.

A coating can be deployed using any aqueous-based method, such as by an aqueous-borne spray-on coating. The spray-on coating can inactivate proteins or virion particles and degrade DNA or RNA of the virus. Without being bound by theory, it is believed that the polymer coating binds and, through subsequent large conformational changes of the nanoworm the nanoworm ruptures the viral membrane. Subsequently, the polymer coating binds and degrades the RNA of the virus, inactivating the virus, such as SARS-CoV-2 (VIC01) and an evolved B.1.1.7 (alpha) variant, influenza A and a surrogate capsid pseudovirus expressing the influenza A virus attachment glycoprotein, hemagglutinin. A polygalactose functionality on a polymer targets the conserved S2 subunit on the SARS-CoV-2 virion surface spike glycoprotein for stronger binding, and the additional attachment of the guanidine or diguanidine groups is known to catalyze the degradation of the RNA genome of the virus.

In some examples, a polymer of the present disclosure may be coated onto a surface of an item of personal protective equipment, such as a mask, a face shield, a rebreather, a filter cartridge, protective eyewear, or combinations thereof. Coating surgical masks with the polymers may result in complete inactivation of VICO1 and B.1.1.7, providing a powerful control measure for SARS-CoV-2 and its variants. Inactivation may be observed for the influenza A and an AAV-HA capsid pseudovirus, providing broad viral inactivation when using a polymer of the present disclosure. The technology described herein represents a coating with a proposed mechanism for inactivation of viruses both enveloped and capsid. Functional polymers can be modified to target other viruses known and unknown, and are compatible with large scale manufacturing processes.

In certain aspects, a functional polymer, according to the present disclosure, coated surface is or can become a hydrophilic surface. For example, a polymer coated surface is or can become hydrophilic (water soluble) allowing the wetting of a droplet, such as a mucosal drop, blood, urine, sweat, other bodily fluids, and other non-bodily fluids, across the polymer coated surface. In certain aspects, pathogens on the surface of the droplet or suspended within the droplet can be captured, inactivated, or deactivated by the polymer coated surface. The coatings described herein include a polymer and may have a transparent appearance when applied to surfaces. In some aspects, the coatings are useful for inactivating one or more, such as all variants of SARS-CoV-2. Without being bound by theory, it is believed that the functional polymer described herein of the coating targets the highly glycosylated spike protein and/or the influenza A virus attachment glycoprotein, hemagglutinin, on a virion surface and disrupt the viral membrane through a process of binding via hydrophobic functional groups to perform a mechanical rupture of the virus membrane.

Conventional polymers can have a non-functional group to assist with stability during the synthesis. However, a polymer of the present disclosure may avoid the use of non-functional groups, such as styrene, as compared to conventional antipathogenic coatings. to avoid agglomeration and reduce a binding efficacy of the polymer to the virion. In some aspects, the polymer may be substantially free of non-functional groups, such as styrene.

In certain aspects, personal protective equipment (e.g. a face mask, a face shield, a rebreather, a filter cartridge, protective eyewear, or combinations thereof) and treatment of high-touch surfaces with antiviral coatings of the present disclosure can provide long-lasting (e.g., days, weeks, months, etc.) disinfection of contaminated surfaces to reduce or eliminate the spread of SARS-CoV-2 and/or variants thereof.

### Definitions

The term “DNA” refers to a polymer composed of two polynucleotide chains that coil around each other to form a double helix. DNA, otherwise known as deoxyribonucleic acid includes one or more of adenine, cytosine, guanidine, and/or thymine. DNA may include a modified base, e.g., 5-methylcytosine, N₆-carbamoyl-methyladenine, N₆-methadenine, 7-Deazaguanine, 7-Methylguanine, N₄-methylcytosine, 5-carboxylcytosine, 5-formylcytosine, 5-glycosylhydroxymethylcytosine, 5-hydroxycytosine, 5-methylcytosine, alpha-glutamythymidine, alpha-putrrescinylthymine, base j, uracil, 5-dihydroxypentauracil, 5-hydroxymethyldeoxyuracil, deoxyarchaeosine, 2,6-diaminopurine, or combinations thereof. A DNS virus may include a double-stranded DNA virus or a single-stranded DNA virus.

As used herein, the term “pathogen” refers to viruses, bacteria, fungi, and/or other microbes or germs. The coatings described herein are capable of reducing or eliminating the presence of and/or transmission of a wide range of pathogens, such as SARS-CoV-2 and variants thereof, such as alpha, beta, delta, omicron, or combinations thereof.

The term “pharmaceutically-acceptable” means suitable for use in pharmaceutical preparations, generally considered as safe for such use, officially approved by a regulatory agency of a national or state government for such use, or being listed in the U.S. Pharmacopoeia or other generally recognized pharmacopoeia for use in animals, and more particularly in humans.

The term “pharmaceutically-acceptable salt” refers to a salt which may enhance desired pharmacological activity. Examples of pharmaceutically-acceptable salts include acid addition salts formed with inorganic or organic acids, metal salts and amine salts. Examples of acid addition salts formed with inorganic acids include salts with hydrochloric acid, hydrobromic acid, sulfuric acid, nitric acid and phosphoric acid. Examples of acid addition salts formed with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxy-benzoyl)-benzoic acid, cinnamic acid, mandelic acid, methanesulfonic acid, ethanesulfonic acid, 1,2-ethanedisulfonic acid, 2-hydroxyethane-sulfonic acid, benzenesulfonic acid, p-chlorobenzenesulfonic acid, 2-naphthalenesulfonic acid, p-toluenesulfonic acid, camphorsulfonic acid, 4-methyl-bicyclo[2.2.2]oct-2-enel-carboxylic acid, gluco-heptonic acid, 4,4′-methylenebis(3-hydroxy-2-naphthoic) acid, 3-phenylpropionic acid, trimethyl-acetic acid, tertiary butylacetic acid, lauryl sulfuric acid, gluconic acid, glutamic acid, hydroxy-naphthoic acids, salicylic acid, stearic acid and muconic acid. Examples of metal salts include salts with sodium, potassium, calcium, magnesium, aluminum, iron, and zinc ions. Examples of amine salts include salts with ammonia and organic nitrogenous bases strong enough to form salts with carboxylic acids.

The term “RNA” refers to a ribonucleic acid living within a virus or cell. RNA may include one or more of adenine, cytosine, guanidine, and/or uracil. RNA may include a phosphate group attached at a 3′ position of the one ribose and the 5′ position of the next. RNA may be a sense RNA. The term “Sense RNA virus” refers to a type of virus containing a positive sense single-stranded RNA or a negative sense single-stranded RNA. A Sense RNA virus may be capable of operating as mRNA and can be directly translated into the protein in the host.

The term “therapeutically-effective amount” refers to an amount of a compound that, when administered to a subject for treating a condition, is sufficient to effect treatment for the condition. “Therapeutically effective amount” can vary depending on the compound, the condition and its severity, the age, and the weight of the subject to be treated.

The term “virus” refers to a submicroscopic infective agent that includes a nonliving complex molecule that typically contains a protein coat surrounding an RNA or DNA core of genetic material but no semipermeable membrane, that is capable of growth and multiplication in living cells, and that can cause a disease in humans, animals, or plants. In one, the virus may be a sense RNA virus, e.g., a positive sense RNA virus or a negative sense virus. For example, the virus may be SARS-CoV-2, or any variant thereof.

Compounds of the present disclosure include tautomeric, geometric or stereoisomeric forms of the compounds. Ester, oxime, onium, hydrate, solvate and N-oxide forms of a compound are also embraced by the present disclosure. The present disclosure considers all such compounds, including cis- and trans-geometric isomers (Z- and E-geometric isomers), R- and S-enantiomers, diastereomers, d-isomers, 1-isomers, atropisomers, epimers, conformers, rotamers, mixtures of isomers and racemates thereof are embraced by the present disclosure.

As used herein, the term “SARS-CoV-2 variant” refers to viruses that have mutated from SARS-CoV-2. The mutations can include about 1 to about 75 mutations across the virus genome, such as about 25 to about 50 mutations. One or more the mutations can include mutations in the spike protein of the virus, such as about 1 to about 40 mutations in the spike protein, such as about 32 mutations. Without being bound by theory, it is believed that certain known variants have enhanced binding to the ACE2 receptor through the receptor binding domain on the spike protein found predominantly on human throat and lung cells. Once bound to the cell, the mutation close to the S1/S2 region further enhances cleavage mainly by the serine proteinase (e.g., TMPRSS2) on the cell surface, exposing the spike's hydrophobic region to fuse and release the viral RNA within the cell, or enhance cell-cell fusion of giant multi-nuclear cells. Different variants can have different responses to vaccination, different rates of transmission, and different symptoms upon contraction. An antigenic shift, due to the high number of mutations in certain variants, such as the omicron spike, may stem from extensive replication in immune-deficient hosts or transmissions back and forth between humans and rodents. In some aspects, infected hosts can release SARS-CoV-2 into the environment via sneezing, coughing and skin contact, resulting in potential fomite contamination of surrounding surfaces. Infectious SARS-CoV-2 has been proven in laboratory-based studies to persist on many different surfaces.

### Compound

In at least one aspect, a composition includes a polymer. The polymer includes a plurality of N-alkylacrylamide units. In at least one aspect, N-alkylacrylamide is represented by the formula:

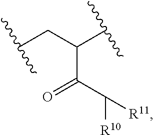

wherein each of R¹⁰ and R¹¹ is independently hydrogen or C₁-C₂₀ alkyl, where at least one of R¹⁰ or R¹¹ is C₁-C₂₀ alkyl, such as methyl, ethyl, n-propyl, or isopropyl. In at least one aspect, at least one of R¹⁰ or R¹¹ is isopropyl.

In at least one aspect, the compound comprises a moiety represented by the formula

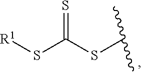

where R¹ is C₁-C₂₀ alkyl. In some aspects, R¹ is methyl, ethyl, propyl, butyl, pentyl, hexyl, heptyl, octyl, nonyl, decyl, or the like. In some aspects, R¹ is butyl.

In at least one aspect, the compound comprises a moiety represented by the formula

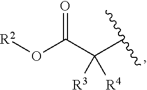

where R², R³, and R⁴ are each independently hydrogen or C₁-C₂₀ alkyl. In some aspects, R², R³, and R⁴ are each independently hydrogen. In some aspects, R², R³, and R⁴ are each independently C₁-C₂₀ alkyl, e.g., methyl, ethyl, propyl, butyl, pentyl, hexyl, heptyl, octyl, nonyl, decyl, or the like. In some aspects, R² and R³ are hydrogen and R⁴ is C₁-C₂₀ alkyl, e.g., methyl, ethyl, propyl, butyl, pentyl, hexyl, heptyl, octyl, nonyl, decyl, or the like. In some aspects, R² and R⁴ are hydrogen and R³ is C₁-C₂₀ alkyl, e.g., methyl, ethyl, propyl, butyl, pentyl, hexyl, heptyl, octyl, nonyl, decyl, or the like. In some aspects, R³ and R⁴ are hydrogen and R² is C₁-C₂₀ alkyl, e.g., methyl, ethyl, propyl, butyl, pentyl, hexyl, heptyl, octyl, nonyl, decyl, or the like. In some aspects, R² is hydrogen and R³ and R⁴ are independently C₁-C₂₀ alkyl, e.g., methyl, ethyl, propyl, butyl, pentyl, hexyl, heptyl, octyl, nonyl, decyl, or the like. In some aspects, R³ is hydrogen and R² and R⁴ are independently C₁-C₂₀ alkyl, e.g., methyl, ethyl, propyl, butyl, pentyl, hexyl, heptyl, octyl, nonyl, decyl, or the like. In some aspects, R² and R⁴ are methyl, and R³ is hydrogen. In some aspects, R⁴ is hydrogen and R² and R³ are independently C₁-C₂₀ alkyl, e.g., methyl, ethyl, propyl, butyl, pentyl, hexyl, heptyl, octyl, nonyl, decyl, or the like. In some aspects, R² and R³ are methyl, and R⁴ is hydrogen.

In at least one aspect, the compound comprises at least two moieties represented by

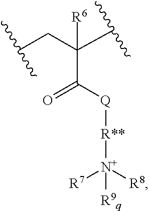

where Q is O or N, R** is C₁-C₂₀ alkyl, e.g., methyl, ethyl, propyl, butyl, pentyl, hexyl, heptyl, octyl, nonyl, decyl, or the like, R⁶, R⁷, and R⁸ are independently C₁-C₆ alkyl or hydrogen. In some aspects, R⁶, R⁷, and R⁸ are each independently C₁-C₆ alkyl, e.g., methyl, ethyl, propyl, butyl, pentyl, hexyl, or the like. In some aspects, R⁶, R⁷, and R⁸ are each methyl. In some aspects, R⁶ and R⁷ are hydrogen and R⁸ is C₁-C₆ alkyl, e.g., methyl, ethyl, propyl, butyl, pentyl, hexyl, or the like. In some aspects, R⁶ and R⁸ are hydrogen and R⁷ is C₁-C₆ alkyl, e.g., methyl, ethyl, propyl, butyl, pentyl, hexyl, or the like. In some aspects, R⁷ and R⁸ are hydrogen and R⁶ is C₁-C₆ alkyl, e.g., methyl, ethyl, propyl, butyl, pentyl, hexyl, or the like. In some aspects, R⁶ is hydrogen and R⁷ and R⁸ are independently C₁-C₆ alkyl, e.g., methyl, ethyl, propyl, butyl, pentyl, hexyl, or the like. In some aspects, R⁷ is hydrogen and R⁶ and R⁸ are independently C₁-C₆ alkyl, e.g., methyl, ethyl, propyl, butyl, pentyl, hexyl, or the like. In some aspects, R⁸ is hydrogen and R⁶ and R⁷ are independently C₁-C₆ alkyl, e.g., methyl, ethyl, propyl, butyl, pentyl, hexyl, or the like.

In some aspects, q is 0 or 1. In some aspects, at least one instance of R⁹ (of the plurality) is C₁-C₁₆ alkyl, e.g., methyl, ethyl, propyl, butyl, pentyl, hexyl, heptyl, octyl, nonyl, decyl, or the like. Without being bound by theory, when R⁹ is C₅ or greater, enhanced cell membrane penetration of the alkyl moiety into the hydrophobic portion of a cell membrane (such as a viral cell) may occur promoting lysis of the viral cell. Additionally, the cationic nitrogen moieties may provide coulombic interactions of the compound (such as the quaternary ammonium moieties) with the cell membrane surface (such as the phosphate moieties of the phospholipid bilayer) further promoting lysis of the viral cell. Furthermore, the quaternary ammonium salt may provide sufficient hydrophilicity so that the alkyl moieties attached to the quaternary ammonium salt do not become substantially buried within the core of the three dimensional structure (e.g., when the composition has the three dimensional structure of nanoworm or nanorod).

In some aspects, R⁹ is C₁-C₆ alkylyne, e.g., C₁ alkylyne, C₂ alkylyne, C₃ alkylyne, C₄ alkylyne, C₅ alkylyne, C₆ alkylyne, or the like. In some aspects, R⁹ is azole. In some aspects, R⁹ is an oligomer of guanidine, e.g., guanidine or diguanidine. In some aspects, R⁹ is a diguanidine represented by the structure:

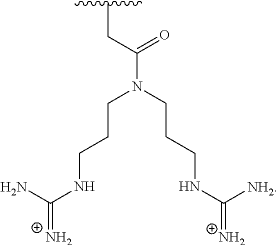 [SMILES: *CC(=O)N(CCCNC(N)=[NH2+])CCCNC(N)=[NH2+]]

In some aspects, R⁹ is polygalactose having the structure:

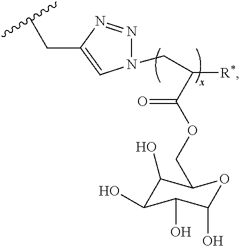

wherein x is an integer of 1 to 20, e.g., an integer of 10, and R* is hydrogen, —OH, or

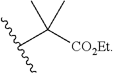 [SMILES: *C(C)(C)C(=O)OCC]

In some aspects, R⁹ is coumarin, such as 7-hydroxycoumarin. In some aspects, R⁹ is a combination of azole and polygalactose (e.g., polygalactose-substituted azole). In some aspects, R⁹ is a combination of azole and coumarin (e.g., 3-azido-7-hydroxycoumarin), having the formula:

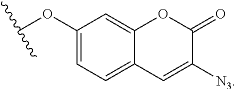 [SMILES: *Oc1ccc2cc(N=[N+]=[N-])c(=O)oc2c1]

In at least one aspect, q is 1 and R⁹ is diguanidine.

For example, the two at least two moieties are represented by the formula:

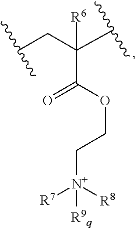

where R⁶, R⁷, and R⁸ are C₁-C₆ alkyl or hydrogen. In some aspects, R⁶, R⁷, and R⁸ are each independently C₁-C₆ alkyl, e.g., methyl, ethyl, propyl, butyl, pentyl, hexyl, or the like. In some aspects, R⁶, R⁷, and R⁸ are each methyl. In some aspects, R⁶ and R⁷ are hydrogen and R⁸ is C₁-C₆ alkyl, e.g., methyl, ethyl, propyl, butyl, pentyl, hexyl, or the like. In some aspects, R⁶ and R⁸ are hydrogen and R⁷ is C₁-C₆ alkyl, e.g., methyl, ethyl, propyl, butyl, pentyl, hexyl, or the like. In some aspects, R⁷ and R⁸ are hydrogen and R⁶ is C₁-C₆ alkyl, e.g., methyl, ethyl, propyl, butyl, pentyl, hexyl, or the like. In some aspects, R⁶ is hydrogen and R⁷ and R⁸ are C₁-C₆ alkyl, e.g., methyl, ethyl, propyl, butyl, pentyl, hexyl, or the like. In some aspects, R⁷ is hydrogen and R⁶ and R⁸ are C₁-C₆ alkyl, e.g., methyl, ethyl, propyl, butyl, pentyl, hexyl, or the like. In some aspects, R⁸ is hydrogen and R⁶ and R⁷ are C₁-C₆ alkyl, e.g., methyl, ethyl, propyl, butyl, pentyl, hexyl, or the like.

In some aspects, R⁹ is C₁-C₁₆ alkyl, e.g., methyl, ethyl, propyl, butyl, pentyl, hexyl, heptyl, octyl, nonyl, decyl, or the like. Without being bound by theory, when R⁹ is C₅ or greater, enhanced cell membrane penetration of the alkyl moiety into the hydrophobic portion of a cell membrane (such as a viral cell) may occur. Additionally, the cationic nitrogen moieties may provide coulombic interactions of the compound (such as the quaternary ammonium moieties) with the cell membrane surface (such as the phosphate moieties of the phospholipid bilayer). Furthermore, the quaternary ammonium salt may provide sufficient hydrophilicity so that the alkyl moieties attached to the quaternary ammonium salt do not become substantially buried within the polymer.

In some aspects, R⁹ is C₁-C₆ alkylyne, e.g., C₁ alkylyne, C₂ alkylyne, C₃ alkylyne, C₄ alkylyne, C₅ alkylyne, C₆ alkylyne, or the like. In some aspects, R⁹ is azole. In some aspects, R⁹ is azole. In some aspects, R⁹ is a guanidine or an oligomer of guanidine, e.g., diguanidine. In some aspects, R⁹ is a diguanidine represented by the structure:

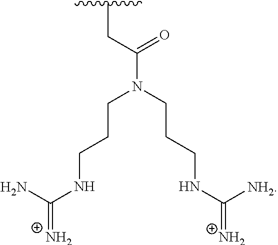 [SMILES: *CC(=O)N(CCCNC(N)=[NH2+])CCCNC(N)=[NH2+]]

In some aspects, R⁹ is polygalactose having the structure:

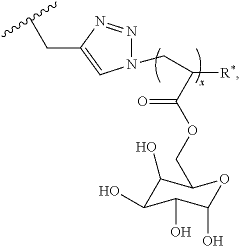

wherein x is an integer of 1 to 20, e.g., an integer of 10, and R* is hydrogen, —OH, or

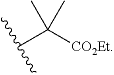 [SMILES: *C(C)(C)C(=O)OCC]

In some aspects, R⁹ is coumarin, such as 7-hydroxycoumarin. In some aspects, R⁹ is a combination of azole and polygalactose (e.g., polygalactose-substituted azole). In some aspects, R⁹ is a combination of azole and coumarin (e.g., 3-azido-7-hydroxycoumarin), having the formula:

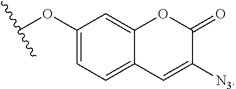 [SMILES: *Oc1ccc2cc(N=[N+]=[N-])c(=O)oc2c1]

In at least one aspect, q is 1.

In at least one aspect, the compound is represented by formula (I):

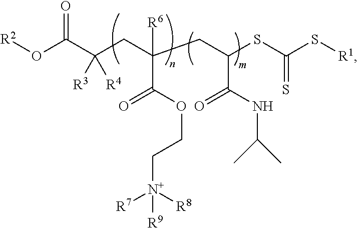

or a pharmaceutically acceptable salt thereof, where n and m are each independently integers of 1 to 100. In some aspects, n is an integer from 1 to 100, e.g., 20 to 50, 25 to 35, or the like. In some aspects, n is 30. In some aspects, m is an integer from 1 to 100, e.g., 30 to 60, 35 to 55, or the like. In some aspects, n is 45. Each instance of q is independently an integer of 0 or 1.

In some aspects, R², R³, and R⁴ are each independently hydrogen. In some aspects, R², R³, and R⁴ are each independently C₁-C₂₀ alkyl, e.g., methyl, ethyl, propyl, butyl, pentyl, hexyl, heptyl, octyl, nonyl, decyl, or the like. In some aspects, R² and R³ are hydrogen and R⁴ is C₁-C₂₀ alkyl, e.g., methyl, ethyl, propyl, butyl, pentyl, hexyl, heptyl, octyl, nonyl, decyl, or the like. In some aspects, R² and R⁴ are hydrogen and R³ is C₁-C₂₀ alkyl, e.g., methyl, ethyl, propyl, butyl, pentyl, hexyl, heptyl, octyl, nonyl, decyl, or the like. In some aspects, R³ and R⁴ are hydrogen and R² is C₁-C₂₀ alkyl, e.g., methyl, ethyl, propyl, butyl, pentyl, hexyl, heptyl, octyl, nonyl, decyl, or the like. In some aspects, R² is hydrogen and R³ and R⁴ are independently C₁-C₂₀ alkyl, e.g., methyl, ethyl, propyl, butyl, pentyl, hexyl, heptyl, octyl, nonyl, decyl, or the like. In some aspects, R³ is hydrogen and R² and R⁴ are independently C₁-C₂₀ alkyl, e.g., methyl, ethyl, propyl, butyl, pentyl, hexyl, heptyl, octyl, nonyl, decyl, or the like. In some aspects, R² and R⁴ are methyl, and R³ is hydrogen. In some aspects, R⁴ is hydrogen and R² and R³ are independently C₁-C₂₀ alkyl, e.g., methyl, ethyl, propyl, butyl, pentyl, hexyl, heptyl, octyl, nonyl, decyl, or the like. In some aspects, R² and R³ are methyl, and R⁴ is hydrogen.

In some aspects, R⁶, R⁷, and R⁸ are each independently C₁-C₆ alkyl, e.g., methyl, ethyl, propyl, butyl, pentyl, hexyl, or the like. In some aspects, R⁶, R⁷, and R⁸ are each methyl. In some aspects, R⁶ and R⁷ are hydrogen and R⁸ is C₁-C₆ alkyl, e.g., methyl, ethyl, propyl, butyl, pentyl, hexyl, or the like. In some aspects, R⁶ and R⁸ are hydrogen and R⁷ is C₁-C₆ alkyl, e.g., methyl, ethyl, propyl, butyl, pentyl, hexyl, or the like. In some aspects, R⁷ and R⁸ are hydrogen and R⁶ is C₁-C₆ alkyl, e.g., methyl, ethyl, propyl, butyl, pentyl, hexyl, or the like. In some aspects, R⁶ is hydrogen and R⁷ and R⁸ are independently C₁-C₆ alkyl, e.g., methyl, ethyl, propyl, butyl, pentyl, hexyl, or the like. In some aspects, R⁷ is hydrogen and R⁶ and R⁸ are independently C₁-C₆ alkyl, e.g., methyl, ethyl, propyl, butyl, pentyl, hexyl, or the like. In some aspects, R⁸ is hydrogen and R⁶ and R⁷ are independently C₁-C₆ alkyl, e.g., methyl, ethyl, propyl, butyl, pentyl, hexyl, or the like.

In some aspects, q is 0 or 1. In some aspects, R⁹ (of the plurality) is C₁-C₁₆ alkyl, e.g., methyl, ethyl, propyl, butyl, pentyl, hexyl, heptyl, octyl, nonyl, decyl, or the like. In some aspects, R⁹ is C₁-C₆ alkylyne, e.g., C₁ alkylyne, C₂ alkylyne, C₃ alkylyne, C₄ alkylyne, C₅ alkylyne, C₆ alkylyne, or the like. In some aspects, R⁹ is azole. In some aspects, R⁹ is a guanidine or an oligomer of guanidine, e.g., diguanidine. In some aspects, R⁹ is a diguanidine represented by the structure:

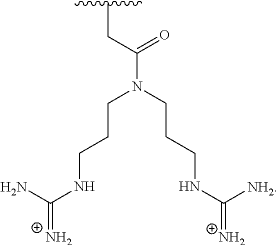 [SMILES: *CC(=O)N(CCCNC(N)=[NH2+])CCCNC(N)=[NH2+]]

In some aspects, R⁹ is a polysachharide, such as a polygalactose. In some aspects,

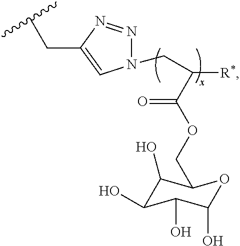

wherein x is an integer of 1 to 20, e.g., an integer of 10, and R* is hydrogen, —OH, or

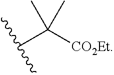 [SMILES: *C(C)(C)C(=O)OCC]

In some aspects, R⁹ is coumarin, such as 7-hydroxycoumarin. In some aspects, R⁹ is a combination of azole and polygalactose (e.g., polygalactose-substituted azole). In some aspects, R⁹ is a combination of azole and coumarin (e.g., 3-azido-7-hydroxycoumarin), having the formula:

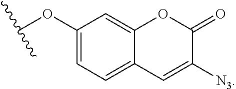 [SMILES: *Oc1ccc2cc(N=[N+]=[N-])c(=O)oc2c1]

In at least one aspect, q is 1.

In some aspects, the polymer may be represented by formula (II):

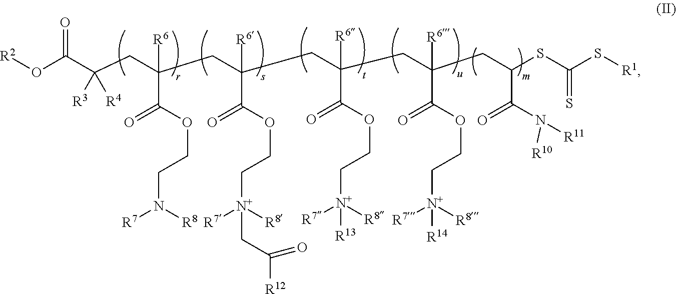

or a pharmaceutically acceptable salt thereof. For example, each of r, s, t, u, and m, are independently 1-100. In some aspects, r is an integer of 1 to 100, e.g., 5 to 40, 5 to 15, or the like. In some aspects, r is 11 or 12. In some aspects, s is an integer of 1 to 100, e.g., 5 to 40, 5 to 15, or the like. In some aspects, s is 11 or 12. In some aspects, t is an integer of 1 to 100, e.g., 1 to 20, 1 to 5, or the like. In some aspects, t is 3 or 4. In some aspects, u is an integer of 1 to 100, e.g., 1 to 20, 1 to 5, or the like. In some aspects, u is 3 or 4. In some aspects, m is an integer of 1 to 100, e.g., 25 to 60, 35 to 55, or the like. In some aspects, m is 45.

In some aspects, R¹ is hydrogen or C₁-C₂₀ alkyl. In some aspects R¹ is butyl. In some aspects, R², R³, and R⁴ are each independently hydrogen. In some aspects, R², R³, and R⁴ are each independently C₁-C₂₀ alkyl, e.g., methyl, ethyl, propyl, butyl, pentyl, hexyl, heptyl, octyl, nonyl, decyl, or the like. In some aspects, R² and R³ are hydrogen and R⁴ is C₁-C₂₀ alkyl, e.g., methyl, ethyl, propyl, butyl, pentyl, hexyl, heptyl, octyl, nonyl, decyl, or the like. In some aspects, R² and R⁴ are hydrogen and R³ is C₁-C₂₀ alkyl, e.g., methyl, ethyl, propyl, butyl, pentyl, hexyl, heptyl, octyl, nonyl, decyl, or the like. In some aspects, R³ and R⁴ are hydrogen and R² is C₁-C₂₀ alkyl, e.g., methyl, ethyl, propyl, butyl, pentyl, hexyl, heptyl, octyl, nonyl, decyl, or the like. In some aspects, R² is hydrogen and R³ and R⁴ are independently C₁-C₂₀ alkyl, e.g., methyl, ethyl, propyl, butyl, pentyl, hexyl, heptyl, octyl, nonyl, decyl, or the like. In some aspects, R³ is hydrogen and R² and R⁴ are independently C₁-C₂₀ alkyl, e.g., methyl, ethyl, propyl, butyl, pentyl, hexyl, heptyl, octyl, nonyl, decyl, or the like. In some aspects, R² and R⁴ are methyl, and R³ is hydrogen. In some aspects, R⁴ is hydrogen and R² and R³ are independently C₁-C₂₀ alkyl, e.g., methyl, ethyl, propyl, butyl, pentyl, hexyl, heptyl, octyl, nonyl, decyl, or the like. In some aspects, R² and R³ are methyl, and R⁴ is hydrogen.

In some aspects, each of R⁶, R^{6′}, R^{6″}, and R^{6′″} are each independently C₁-C₆ alkyl, e.g., methyl, ethyl, propyl, butyl, pentyl, hexyl, or the like. In some aspects, R⁶, R^{6′}, R^{6″}, and R^{6′″} are each methyl. In some aspects, each of R⁷, R^{7′}, R^{7″}, and R^{7′″} are each independently C₁-C₆ alkyl, e.g., methyl, ethyl, propyl, butyl, pentyl, hexyl, or the like. In some aspects, R⁷, R^{7′}, R^{7″}, and R^{7′″} are each methyl. In some aspects, each of R⁸, R^{8′}, R^{8″}, and R^{8′}″ are each independently C₁-C₆ alkyl, e.g., methyl, ethyl, propyl, butyl, pentyl, hexyl, or the like. In some aspects, R⁸, R^{8′}, R^{8″}, and R^{8′″} are each methyl.

In some aspects each of R¹⁰ and R¹¹ is independently hydrogen or C₁-C₂₀ alkyl, where at least one of R¹⁰ or R¹¹ is C₁-C₂₀ alkyl, such as methyl, ethyl, n-propyl, or isopropyl. In at least one aspect, at least one of R¹⁰ or R¹¹ is isopropyl.

In some aspects, R¹² is a guanidine. In some aspects, R¹² is an oligomer of guanidine, e.g., diguanidine. In some aspects, R¹² is a diguanidine represented by the structure:

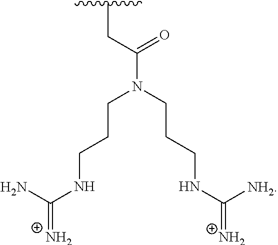 [SMILES: *CC(=O)N(CCCNC(N)=[NH2+])CCCNC(N)=[NH2+]]

In some aspects, R¹³ is C₁-C₆ alkylyne, e.g., C₁ alkylyne, C₂ alkylyne, C₃ alkylyne, C₄ alkylyne, C₅ alkylyne, C₆ alkylyne, or the like. In some aspects R¹³ is C₃ alkylyne. In some aspects, R¹³ is a combination of azole and polygalactose (e.g., polygalactose-substituted azole) having the formula: polygalactose has the structure:

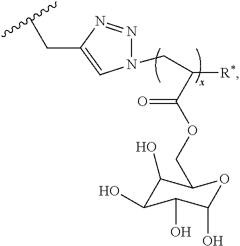

wherein x is an integer of 1 to 20, e.g., an integer of 10, and R* is hydrogen, —OH, or

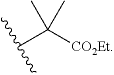 [SMILES: *C(C)(C)C(=O)OCC]

In some aspects, R¹³ is a combination of azole and coumarin (e.g., 3-azido-7-hydroxycoumarin), having the formula:

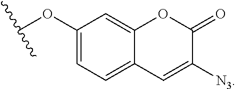 [SMILES: *Oc1ccc2cc(N=[N+]=[N-])c(=O)oc2c1]

In some aspects, at least one instance of R¹³ (of the plurality) is C₁-C₁₆ alkyl, e.g., methyl, ethyl, propyl, butyl, pentyl, hexyl, heptyl, octyl, nonyl, decyl, or the like. In some aspects, R¹³ is octyl.

### Polymer Coating

In some aspects, the polymer may include alkene units and macroCTA polymer units. The polymer includes functional groups from the macroCTA polymer units. Each of the functional groups is a component from a reversible addition-fragmentation chain-transfer (RAFT) agent, which can be pre-functionalized or post-functionalized. In some examples, each of the functional groups is selected to modify the capture and inactivation/deactivation efficiency of the polymer and/or to modify the responsiveness (e.g., temperature, pH, salinity concentration, light, and/or combinations thereof) of the polymer.

### Synthesis of the Polymer

Methods of forming a polymer is provided. In some aspects, the polymer can be produced directly in water using an emulsion polymerization method. The method includes introducing, in a reactor, (1) a first compound having N-alkylacrylamide units and (2) a second compound having N,N-(dialkylamino)(divalent alkyl)alkylacrylate units and N-alkylacrylamide units to form a mixture. In some aspects, introducing the first polymer having N-alkylacrylamide units and the second polymer having N,N-(dialkylamino)(divalent alkyl)alkylacrylate units and N-alkylacrylamide units is performed at a temperature of about −10° C. to about 10° C.

The first polymer may consist of: (1) the N-alkylacrylamide units as N-isopropylacrylamide units, (2) a moiety represented by the formula:

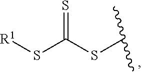

where R¹ is alkyl, and (3) a moiety represented by the formula:

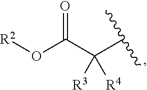

where R² is alkyl (branched or linear, substituted or unsubstituted) and R³ and R⁴ are independently hydrogen or alkyl (branched or linear, substituted or unsubstituted).

In some aspects, the first polymer is free of N,N-(dialkylamino)(divalent alkyl)alkylacrylate units. The second polymer consists of (1) the N,N-(dialkylamino)(divalent alkyl)alkylacrylate units as N,N-(dimethylamino)ethyl methacrylate units, the N-alkylacrylamide units as N-isopropylacrylamide units, a moiety represented by the formula:

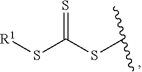

where R¹ is alkyl, and (2) a moiety represented by the formula:

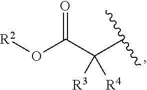

where R² is alkyl (branched or linear, substituted or unsubstituted) and R³ and R⁴ are independently hydrogen or alkyl (branched or linear, substituted or unsubstituted).

The method can include introducing an initiator compound to the mixture to form a second mixture having the polymer. The initiator compound is a peroxide, a hydroperoxide, or an azo initiator. In some examples, the initiator is azobisisobutyronitrile.

The polymer can be coupled with a variety of functional groups, including the hydrophobic octane (O), guanidine (G), diguanidine (DG), a fluorescence probe (C) (e.g., coumarin), and polysaccharide, e.g., polygalactose(S). Binding to the highly glycosylated spike S protein targeted through (i) the strong multivalent binding with polygalactose, and (ii) electrostatic interactions between the negatively charged viral particles and the positively charged guanidine and N,N-(dimethylamino)ethyl methacrylate (DMAEMA) groups. The attached octane groups facilitate the rupture of the viral membrane, in which the viral mRNA can either be degraded by the diguanidine groups or electrostatically captured by the polymer coating. The polymers described herein can be coated on surfaces, including a surgical mask, and readily inactivate sense RNA viruses, e.g., the influenza A virus, ancestral SARS-CoV-2 isolate, alpha variant, and omicron variant.

In certain aspects, the polymer includes a copolymer of a macro chain transfer agent (macroCTA) polymer units and alkene units. A macroCTA polymer is a polymer formed by RAFT using a RAFT agent in the polymerization of one or more ethylenically unsaturated monomers.

In some examples, two macro-chain transfer RAFT poly(N-isopropylacrylamide) (PNIPAM) agents may be produced from a single non-functional RAFT agent. The emulsion polymerization using the two macro-chain transfer agents (e.g., initiated by azobisisobutyronitrile (AIBN) at 70° C. in a 500 mL reactor) may produce polymers consisting of two block copolymers of MacroCTAs A and B at an approximately 8 wt % of polymer in water.

The polymer can then be coupled to the functional groups (O, G) via quaternization, dialyzed, freeze-dried and rehydrated with water to make a 1.5 wt % polymer/water dispersion. The polymer may then be coupled to the functional groups (S and C), via a copper catalyzed azide alkyne cycloaddition (CuAAC) reaction. The CuAAC may use a combination of CuSO₄ and sodium ascorbate. The samples may then be dialyzed, free-dried and rehydrated with water to make a 1.5 wt % polymer/water dispersion. The polymer (NW_{S,O,C,G,DG}) dispersion may then be coated onto surfaces ranging from 1 to 5 sprays. The amount of polymer per area may be determined by measuring the dry weight of polymer on the surface of a glass slide using a microbalance.

### Methods for Depositing Compounds and Compositions

Compounds and compositions of the present disclosure may be deposited onto a surface of an object by any suitable deposition method. A surface of an object may be any suitable surface of any suitable object. A surface may be porous or nonporous. Deposition methods can include one or more of painting, dipping, spraying, marking, taping, brush coating, spin coating, roll coating, doctor-blade coating. Before deposition, a compound or composition of the present disclosure can be diluted in an aqueous solvent, such as a polar solvent, a protic solvent, an aprotic solvent, or the like, e.g., water. After deposition, the solvent may then evaporate at room temperature forming a compound/composition layer on the object.

In at least one aspect, the object is an interior surface of a building or a transport vehicle, such as an aircraft/spacecraft/boat/train or an air filter surface of an aircraft/spacecraft/boat/train, such as a surface of an air-conditioning or filtration system. The object can be a floor surface, seat surface, tray table surface, overhead bin surface, ceiling surface, trim surface, screen surface, window surface, door surface and/or door handle surface of the interior of building or a vehicle such as in an aircraft.

In at least one aspect, a compound or composition of the present disclosure is applied, (e.g., sprayed, deposited, printed, etc.) onto a surface of an object for about 1 second to about 10 minutes, such as about 30 seconds to about 2 minutes. In at least one aspect, a compound or composition is applied (e.g., sprayed) onto a surface of an object in an amount of about 1 mL to about 25 kL, such as about 100 L to about 1 kL. The compound or composition of the present disclosure can be applied, in which the surface will appear wet due to the solvent of the composition.

Compounds or compositions of the present disclosure disposed on an object prevents, reduces, and/or eliminates the presence of bacteria and viruses (such as SARS-CoV-2), which can prevent, reduce, and/or eliminate human contact with such bacteria and viruses. The compounds or compositions of the present disclosure bind to the bacteria and/or virus as described herein, in which a conformational change of the nanoworm and/or nanorod occurs such that the membrane of the bacteria and/or virus is ruptures causing inactivation of the bacteria and/or virus. The inactivation reduces the amount of human contact with the bacteria and/or virus.

Compositions can have any suitable pH, such as a pH of about 6.5 to about 7.4. For example, a pH of about 6.5 mimics the pH of a mucosal droplet. Without wishing to be bound by theory, the pH of the composition can assist in the antibacterial or antiviral capabilities of the composition, in which a pH that mimics the pH of the mucosal droplet may assist in reducing the presence of a bacteria or virus.

Compositions comprising polymers of the present disclosure are advantageous to deposit onto a surface and/or impregnate a fabric or porous material because, for example, an antibacterial and antiviral compound can be applied as a single layer, maintaining efficacy of both compounds. Applying a composition having a polymer as a single layer also reduces cost and time of applying the compounds to a surface, as compared to application of two or more layers. By using a water-based solution, end-user safety is achieved and thus time savings and cost savings for application to a surface will be realized. Alternatively, in some examples, thicker layers and/or multiple layers may be applied. In some examples, a surface is refreshed or replenished with one or more additional layers of polymer composition at a time after application of a first application (one layer or multiple layers) based on a desired amount of antibacterial or antiviral protection.

In some aspects, methods of disposing a polymer onto a surface are described herein. In some aspects, a method includes depositing the polymer on the surface. In some aspects, a method includes impregnating a fabric or fiber, e.g., woven or non-woven. The polymer includes a compound or salt thereof.

The surface to be treated with the coating can be any suitable surface of any suitable object. In some non-limiting aspects, an object is a mask and a surface is an interior portion of a fuselage of an aircraft, or any other suitable surface. In some examples, a surface is a surface (interior or exterior) of an aircraft, a ship, a train, a terminal (e.g., bus, train, airport, etc.), or a spacecraft.

In some aspects, the solution has a concentration of the polymer of about 0.5 wt % to about 3 wt % of polymer in the total volume of the solution, allowing for a reduction of bacteria or viruses to be achieved.

### Methods for Use as a Pharmaceutical Drug

In some aspects, the present disclosure further provides methods for treating a condition in a subject having or susceptible to having such a condition, by administering to the subject a therapeutically-effective amount of one or more compounds or compositions of the present disclosure. In one aspect, the treatment is preventative treatment. In another aspect, the treatment is palliative treatment. In another aspect, the treatment is restorative treatment.

A method for treating a condition can include administering to a subject a therapeutically effective amount of a polymer, or pharmaceutically acceptable salt thereof (or a composition having a polymer, or pharmaceutically acceptable salt thereof).

Methods for treating a condition are described herein. In some aspects, a method includes administering to a subject a therapeutically effective amount of a polymer.

A coating described herein can be scaled and applied directly to surfaces as a water solution to act as an effective virucidal agent that renders SARS-CoV-2 variants of concern non-infectious. The design of the polygalactose (e.g., about 2 to about 20 galactose units) attached to the polymer nanostructure and potential specific bonding interactions with highly glycosylated SARS-CoV-2 provide a binding motif independent of the virus variant and mutations found in the virus spike attachment glycoprotein. In some aspects, a polygalactose has greater than 20 galactose units, e.g., up to about 1,000 galactose units. The polygalactose binding in combination with the octane moieties and the responsive nature of the nanostructures that mechanically attach to and disrupt the viral particles, rendering them non-infectious. Without being bound by theory, SARS-CoV-2 viral RNA genome may either degrade as a result of the diguanidine groups or be electrostatically captured by the cationic groups attached to the polymer that then allows natural degradation. It has been discovered that the viral RNA genome cannot be detected after interaction of the viruses with the polymer coated surfaces which demonstrates complete virucidal activity of the polymer. It is believed that the polymer coating provides inactivation of newly emerging SARS-CoV-2 variants of concern while still maintaining the ability to be re-designed via functionalization to target other viruses. Finally, the polymer was found to be non-toxic by oral ingestion in rats and had little or no skin sensitization when applied on the skin of mice, indicating the potential safe use as a component of personal protective equipment or high touch-point surfaces that comes into contact with skin. The nanostructure composition can also be administered to subjects as a therapeutic treatment.

### 1. Conditions

The conditions that can be treated in accordance with the present disclosure include, but are not limited to, conditions caused by a toxin (such as an antigen) and inflammatory disorders such as septic shock. The conditions that can be treated in accordance with the present disclosure include, but are not limited to viral infections, bacterial infections, chronic inflammatory disorders, acute inflammatory disorders, and cancers. In some aspects, the condition to be treated includes a bacterial infection, a viral infection, or a cancer immunotherapy. Cancer immunotherapy can include cervical cancers such as those resulting from an infection of the cervix with human papillomavirus.

Viral infections can include those caused by Ebola, influenza, SARS (such as SARS CoV-2), Noro (gastro), or Zika. Viral infections can include viral respiratory infections (e.g., of the nose, throat, upper airways, or lungs) such as pneumonia, laryngotracheobronchitis, bronchiolitis. Viral infections can include viral gastrointestinal infections such as gastroenteritis caused by a norovirus or rotavirus. Viral infections can include viral liver infections such as hepatitis. Viral infections can include viral nervous system infections such as encephalitis caused by rabies or West Nile virus. Viral infections include warts and/or infections caused by human papilloma virus (HPV). Viral infections can include infections that cause cancer such as infections caused by Epstein-Barr virus, Hepatitis B, Hepatitis C, Herpesvirus 8, or Human papillomavirus. Symptoms of viral infections can include fever, muscle aches, coughing, sneezing, runny nose, headache, chills, diarrhea, vomiting, rash, or weakness.

Bacterial infections can include pneumonia, meningitis, food poisoning, and bacterial skin infections such as those caused by Staphylococcus or Streptococcus, cellulitis, folliculitis, impetigo, and boils. Bacterial infections (e.g., by food poisoning) can include infections caused by Escherichia coli (E. coli), Campylobacter jejuni (C. jejuni), Clostridium botulinum (C. botulinum), Listeria monocytogenes (L. monocytogenes), Salmonella, and Vibrio. Bacterial infections can include bacterial meningitis, otitis media, urinary tract infection, and respiratory tract infections such as sore throat, bronchitis, sinusitis, and pneumonia. Symptoms of bacterial infections can include nausea, vomiting, diarrhea, fever, chills, and abdominal pain.

In some aspects, the methods described herein are used to treat patients with disorders arising from dysregulated cytokine, enzymes and/or inflammatory mediator production, stability, secretion, posttranslational processing. Examples of cytokines that may be dysregulated include interleukins 1, 2, 6, 8, 10, 12, 17, 22, and 23 along with tumor necrosis factor alpha and interferons alpha, beta, and gamma. Examples of inflammatory mediators that may be dysregulated include nitric oxide, prostaglandins, and leukotrienes. Examples of enzymes include cyclo-oxygenase, nitric oxide synthase, and matrixmetalloprotease.

Examples of inflammatory conditions relevant to the technology include, but are not limited to, sepsis, septic shock, endotoxic shock, exotoxin-induced toxic shock, gram negative sepsis, and toxic shock syndrome. Inflammatory conditions can include those experienced by immunosuppressed individuals, and can also include “superbugs”, including bacterial and viral strains resistant to current therapeutics.

### 2. Subjects

Suitable subjects to be treated according to the present disclosure include mammalian subjects. Mammals according to the present disclosure include, but are not limited to, human, canine, feline, bovine, caprine, equine, ovine, porcine, rodents, lagomorphs, primates, and the like, and encompass mammals in utero. Subjects may be of either gender and at any stage of development.

### 3. Administration and Dosing

Compounds or compositions of the present disclosure can be administered to a subject in a therapeutically effective amount.

Compounds or compositions of the present disclosure can be administered by any suitable route in the form of a pharmaceutical composition adapted to such a route, and in a dose effective for the treatment intended. An effective dosage is typically in the range of about 0.001 to about 100 mg per kg body weight per day, such as about 0.01 to about 30 mg/kg/day, in single or divided doses. Depending on age, species and condition being treated, dosage levels below the lower limit of this range can be suitable. In other cases, still larger doses can be used without side effects. Larger doses can also be divided into several smaller doses, for administration throughout the day.

### Pharmaceutical Compositions

For the treatment of the conditions referred to above, the compounds described herein can be administered as follows:

### Oral Administration

Compounds or compositions of the present disclosure can be administered orally, including by swallowing, so that the compound enters the gastrointestinal tract, or absorbed into the blood stream directly from the mouth (e.g., buccal or sublingual administration).

Suitable compositions for oral administration include solid formulations such as tablets, lozenges and capsules, which can contain liquids, gels, or powders. Compositions for oral administration may be formulated as immediate or modified release, including delayed or sustained release, optionally with enteric coating.

Liquid formulations can include solutions, syrups and suspensions, which can be used in soft or hard capsules. Such formulations can include a pharmaceutically acceptable carrier, for example, water, ethanol, polyethylene glycol, cellulose, or an oil. The formulation can also include one or more emulsifying agents and/or suspending agents.

In a tablet dosage form the amount of a compound present can be from about 0.05% to about 95% by weight, such as about 2% to about 50% by weight of the dosage form. In addition, tablets may contain a disintegrant, comprising about 0.5% to about 35% by weight, such as about 2% to about 25% of the dosage form. Examples of disintegrants include: methyl cellulose, sodium or calcium carboxymethyl cellulose, croscarmellose sodium, polyvinylpyrrolidone, hydroxypropyl cellulose, or starch.

Suitable lubricants, for use in a tablet, can be present in amounts of about 0.1% to about 5% by weight. Lubricants can include calcium, zinc or magnesium stearate, or sodium stearyl fumarate.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol, or starch.

Suitable surface-active agents and glidants, for use in a tablet, may be present in amounts from about 0.1% to about 3% by weight. Surface active agents and glidants can include polysorbate 80, sodium dodecyl sulfate, talc, or silicon dioxide.

### Parenteral Administration

Compounds and compositions of the present disclosure can be administered directly into the blood stream, muscle, or internal organs. Suitable methods for parenteral administration can include intravenous, intra-muscular, subcutaneous intraarterial, intraperitoneal, intrathecal, or intracranial. Suitable devices for parenteral administration include injectors (including needle and needle-free injectors) and infusion methods.

Compositions for parenteral administration can be formulated as immediate or modified release, including delayed or sustained release.

Most parenteral formulations are aqueous solutions containing excipients, including salts, buffering agents and carbohydrates. A parenteral formulation can include a non-aqueous solution or organic solution containing excipients, including salts, buffering agents and carbohydrates.

Parenteral formulations can also be prepared in a dehydrated form (e.g., by lyophilization) or as sterile non-aqueous solutions. These formulations can include water. Solubility-enhancing agents can also be used in preparation of parenteral solutions.

### Topical Administration

Compounds and compositions of the present disclosure can be administered topically to the skin or transdermally. Formulations for this topical administration can include lotions, solutions, creams, gels, hydrogels, ointments, foams, implants, patches and the like. Pharmaceutically acceptable carriers for topical administration formulations can include water, alcohol, mineral oil, glycerin, polyethylene glycol and the like. Topical administration can be performed by electroporation, iontophoresis, or phonophoresis.

Compositions for topical administration can be formulated as immediate or modified release, including delayed or sustained release.

### Combinations and Combination Therapy

The compounds and compositions of the present disclosure can be used, alone or in combination with other pharmaceutically active compounds, to treat conditions such as those previously described above. The compound(s)/composition(s) of the present disclosure and other pharmaceutically active compound(s) can be administered simultaneously (either in the same dosage form or in separate dosage forms) or sequentially. Accordingly, in one aspect, the present disclosure includes methods for treating a condition by administering to the subject a therapeutically-effective amount of one or more compounds of the present disclosure and one or more additional, different pharmaceutically active compounds.

In another aspect, there is provided a pharmaceutical composition comprising one or more compounds of the present disclosure, one or more additional pharmaceutically active compounds, and a pharmaceutically acceptable carrier.

In another aspect, the one or more additional, different pharmaceutically active compounds is one or more anti-inflammatory drugs, anti-atherosclerotic drugs, immunosuppressive drugs, immunomodulatory drugs, cytostatic drugs, anti-proliferative agents, angiogenesis inhibitors, kinase inhibitors, cytokine blockers, or inhibitors of cell adhesion molecules.

Compounds and compositions of the present disclosure can also be used in combination with other therapeutic reagents that are selected for their therapeutic value for the condition to be treated. In general, the compounds and compositions described herein and, in aspects where combinational therapy is employed, other agents do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified. In certain instances, it is appropriate to administer a compound of the present disclosure as described herein in combination with another, different therapeutic agent. By way of example only, if one of the side effects experienced by a patient upon receiving a compound of the present disclosure is rash, then it is appropriate to administer an anti-histamine agent in combination with the initial therapeutic agent. Or, by way of example only, the therapeutic effectiveness of a compound of the present disclosure is enhanced by administration of another therapeutic agent (which also includes a therapeutic regimen) that also has therapeutic benefit. Regardless of the disease, disorder or condition being treated, the overall benefit experienced by the patient is either simply additive of the two therapeutic agents or the patient experiences a synergistic benefit.

Therapeutically effective dosages vary when the drugs are used in treatment combinations. Methods for experimentally determining therapeutically effective dosages of drugs and other agents for use in combination treatment regimens are documented methodologies. Combination treatment further includes periodic treatments that start and stop at various times to assist with the clinical management of the patient. In any case, the multiple therapeutic agents (one of which is a compound of the present disclosure) are administered in any order, or even simultaneously. If simultaneously, the multiple therapeutic agents are optionally provided in a single, unified form, or in multiple forms (by way of example only, either as a single pill or as two separate pills).

In some aspects, one of the therapeutic agents is given in multiple doses, or both are given as multiple doses. If not simultaneous, the timing between the multiple doses optionally varies from more than zero weeks to less than twelve weeks.

In addition, the combination methods, compositions and formulations are not to be limited to the use of only two agents, the use of multiple therapeutic combinations are also envisioned. It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually used can vary widely, in some aspects, and therefore can deviate from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also administered sequentially, with either agent being administered by a regimen calling for two-step administration. The two-step administration regimen optionally calls for sequential administration of the active agents or spaced-apart administration of the separate active agents. The time period between the multiple administration steps ranges from, a few minutes to several hours, depending upon the properties of each pharmaceutical agent, such as potency, solubility, bioavailability, plasma half-life and kinetic profile of the pharmaceutical agent. Circadian variation of the target molecule concentration is optionally used to determine the optimal dose interval.

Compounds of the present disclosure or compositions having a compound of the present disclosure can be used (e.g., administered) in combination with drugs from the following classes: NSAIDs, immunosuppressive drugs, immunomodulatory drugs, cytostatic drugs, anti-proliferative agents, angiogenesis inhibitors, biological agents, steroids, vitamin D3 analogs, retinoids, other kinase inhibitors, cytokine blockers, corticosteroids and inhibitors of cell adhesion molecules. Where a subject is suffering from or at risk of suffering from atherosclerosis or a condition that is associated with atherosclerosis, a compound or composition of the present disclosure can be optionally used together with one or more agents or methods for treating atherosclerosis or a condition that is associated with atherosclerosis in any combination. Examples of therapeutic agents/treatments for treating atherosclerosis or a condition that is associated with atherosclerosis include, but are not limited to any of the following: torcetrapib, aspirin, niacin, HMG CoA reductase inhibitors (e.g., atorvastatin, fluvastatin, lovastatin, pravastatin, rosuvastatin and simvastatin), colesevelam, cholestyramine, colestipol, gemfibrozil, probucol and clofibrate.

Where a subject is suffering from or at risk of suffering from an inflammatory condition, a compound or composition of the present disclosure is optionally used together with one or more agents or methods for treating an inflammatory condition in any combination. Examples of therapeutic agents/treatments for treating an autoimmune and/or inflammatory condition include, but are not limited to any of the following: corticosteroids, nonsteroidal antiinflammatory drugs (NSAID) (e.g. ibuprofen, naproxen, acetaminophen, aspirin, Fenoprofen (NALFON®), Flurbiprofen (ANSAID®), Ketoprofen, Oxaprozin (DAYPRO®), Diclofenac sodium (VOLTAREN®), Diclofenac potassium (CATAFLAM®), Etodolac (LODINE®), Indomethacin (INDOCIN®), Ketorolac (TORADOL®), Sulindac (CLINORIL®) Tolmetin (TOLECTIN®), Meclofenamate (MECLOMEN®), Mefenamic acid (PONSTEL®), Nabumetone (RELAFEN®), Piroxicam (FELDENE®), cox-2 inhibitors (e.g., celecoxib (CELEBREX®))), immunosuppressants (e.g., methotrexate (RHEUMATREX®), leflunomide (ARAVA®), azathioprine (IMURAN®), cyclosporine (NEORAL®, SANDIMMUNE®), tacrolimus and cyclophosphamide (CYTOXAN®), CD20 blockers (RITUXIMAB®), Tumor Necrosis Factor (TNF) blockers (e.g., etanercept (ENBREL®), infliximab (REMICADE®) and adalimumab (HUMIRA®)), Abatacept (CTLA4-Ig) and interleukin-1 receptor antagonists (e.g. Anakinra (KINERET®), interleukin 6 inhibitors (e.g., ACTEMRA®), interleukin 17 inhibitors (e.g., AIN457), Janus kinase inhibitors (e.g., Tasocitinib), syk inhibitors (e.g. R788), chloroquine and its derivatives.

For use in cancer and neoplastic diseases a compound or composition of the present disclosure is optionally used together with one or more of the following classes of drugs: wherein the anti-cancer agent is an EGFR kinase inhibitor, MEK inhibitor, VEGFR inhibitor, anti-VEGFR2 antibody, KDR antibody, AKT inhibitor, PDK-1 inhibitor, PI3K inhibitor, c-kit/Kdr tyrosine kinase inhibitor, Bcr-Abl tyrosine kinase inhibitor, VEGFR2 inhibitor, PDGFR-beta inhibitor, KIT inhibitor, Flt3 tyrosine kinase inhibitor, PDGF receptor family inhibitor, Flt3 tyrosine kinase inhibitor, RET tyrosine kinase receptor family inhibitor, VEGF-3 receptor antagonist, Raf protein kinase family inhibitor, angiogenesis inhibitor, Erb2 inhibitor, mTOR inhibitor, IGF-IR antibody, NFkB inhibitor, proteosome inhibitor, chemotherapy agent, or glucose reduction agent.

### Examples

Reagents: Unless otherwise stated, all chemicals were used as received. The solvents used were of either HPLC or AR grade; these included dichloromethane (DCM, Aldrich AR grade), DMSO (Aldrich, 99.9%), n-hexane (Emsure, ACS), chloroform (Emsure, ACS), methanol (Merck, Emsure, ACS), acetonitrile (LiChrosolv, hypergrade for LC-MS), petroleum spirit (BR 40-60° C., Univar, AR), toluene (Merck, for analysis EMSURE ACS, ISO, Reag. Ph Eur), ethyl acetate (ChemSupply, AR), ethanol (ChemSupply, AR), N,N-dimethylformamide (DMF: Labscan, AR grade), and N,N-dimethylacetamide (Aldrich, >99%). Activated basic alumina (Aldrich: Brockmann I, standard grade, ~150 mesh, 58 Å), silica gel (Aldrich, 230-400 mesh, 60 Å), magnesium sulphate (anhydrous, Aldrich), Milli-Q water (Biolab, 18.2 MΩm), sodium dodecyl sulphate (SDS, Aldrich, 99%), 1-butanethiol (Aldrich, 99%), D-(+)-galactose (Aldrich, ≥99%), propargyl bromide solution (Aldrich, 80 wt. % in toluene, contains 0.3% magnesium oxide as stabilizer), lithium chloride (Aldrich, 99%), tripotassium phosphate (Aldrich, ≥98%), potassium hydroxide (Aldrich), 3-chloropropylamine hydrochloride (Aldrich, 98%), triethylamine (Aldrich, ≥99.5%), acryloyl chloride (Merck, stabilized with phenothiazine), sodium hydrogen carbonate (Aldrich, 99.5%), sodium azide (Aldrich, ≥99.5%), hydrochloric acid (36%, Ajax, AR), sulfuric acid (Aldrich, 98%), trifluoroacetic acid (Merck, >99%), carbon disulfide (Aldrich, >99.9%), methyl-2-bromopropionate (MBP, Aldrich, 98%), 2-ethyl-2-thiopseudourea hydrobromide (Aldrich, 98%), iodooctane (Aldrich, 98%), copper (II) sulfate (Aldrich, 99%), copper (II) sulfate anhydrous powder (Aldrich, ≥99.99% trace metals basis), Cu(0) powder (Aldrich, <425 μm, 99.5% trace metals basis), and L-ascorbic acid (Aldrich, 99%) were used as received.

Monomers, initiator, and ligand: N-isopropylacrylamide (NIPAM, Aldrich, 97%) and N,N-(dimethylamino)ethyl methacrylate (DMAEMA, Aldrich, 98%) were dissolved in ethanol with activated basic alumina and after filtration used directly for the synthesis of macro chain transfer agents (MacroCTAs). Azobisisobutyronitrile (AIBN, Riedel-de Haen) was recrystallized from methanol twice prior to use. Tris(2-(dimethylamino)ethyl)amine (Me₆TREN),₁ Cu(II)Br₂/Me₆TREN complex,₂ 3-azido-7-hydroxycoumarin azide (coumarin azide)₃ were synthesized according to literature procedures.

RAFT agent: Methyl 2-(butylthiocarbonothioylthio) propanoate (MCEBTTC) RAFT agent was synthesized according to the literature procedure.

Nuclear Magnetic Resonance (NMR) All NMR spectra were recorded on either Bruker DRX 400 or 500 MHz spectrometers using an external lock (CDCl₃, DMSO-d6 or D₂O).

Size Exclusion Chromatography (SEC) and Triple Detection-Size Exclusion Chromatography (TD-SEC): Analysis of the molecular weight distributions of the polymers was determined using a Polymer Laboratories GPC50 Plus equipped with differential refractive index detector. Absolute molecular weights of polymers were determined using a Polymer Laboratories GPC50 Plus equipped with dual angle laser light scattering detector, viscometer, and differential refractive index detector. HPLC grade N,N-dimethylacetamide (DMAc, containing 0.03 wt % LiCl) was used as the eluent at a flow rate of 1.0 mL/min. Separations were achieved using two PLGel Mixed B (7.8× 300 mm) SEC columns connected in series and held at a constant temperature of 50° C. InfinityLab EasiVial polystyrene standards were used for SEC column calibration. Samples of known concentration were freshly prepared in DMAc+0.03 wt % LiCl and passed through a 0.45 μm PTFE syringe filter prior to injection. The absolute molecular weights and dn/dc values were determined using Polymer Laboratories Multi Cirrus software based on the quantitative mass recovery technique.

Synthesis of P(NIPAM-co-DMAEMA) with High Molecular Weights

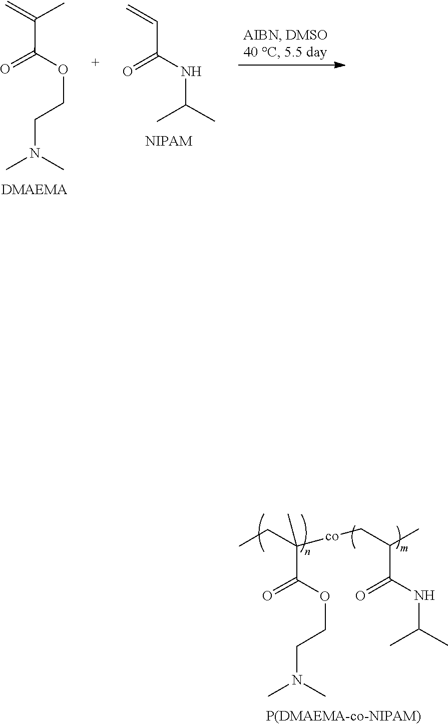

Polymers A and B were synthesized using monomers NIPAM (recrystallized) and DMEMA (inhibitor removed by passing through plug of basic Al₂O₃) according to Tables 1 and 2, respectively.

TABLE 1
Synthesis of polymer A via free radical
polymerization with AIBN at 40 °C.

| Reagent | Mw | mole/mmol | mass/g | density | volume/mL |
|---|---|---|---|---|---|
| NIPAM | 113.16 | 20^{a} | 2.263 | — | — |
| DMAEMA | 157.21 | 10^{a} | 1.572 | 0.944 | 1.67 |
| AIBN | 164.21 | 0.273 | 0.045 | — | — |
| DMSO | — | — | — | — | 2.8 |

^{a}Feeding ratio NIPAM:DMAEMA 2:1

TABLE 2
Synthesis of polymer B via free radical
polymerization with AIBN at 40 °C.

| Reagent | Mw | mole/mmol | mass/g | density | volume/mL |
|---|---|---|---|---|---|
| NIPAM | 113.16 | 8.84^{a} | 1.00 | — | — |
| DMAEMA | 157.21 | 5.34^{a} | 0.84 | 0.944 | 0.89 |
| AIBN | 164.21 | 0.128 | 0.021 | — | — |
| DMSO | — | — | — | — | 1.1 |

^{a}Feeding ratio NIPAM:DMAEMA 3:2

Monomers were mixed with DMSO in a reaction tube. After degassing with argon bubbling for 30 min, the tube was capped with a glass stopper and placed in an oil bath preheated to 40° C. After reaction with 5.5 days, the reaction tube was opened to air and gel-like mixture was dissolved in ice water (24 h dissolving time).

Precipitation by heating up the ice-cold polymer solution was unsuccessful as there was no obvious precipitates formed while the solution became a cloudy latex. So that the polymers were purified via dialysis (MWCO 10 k Da, 5 buffer changes, 24 h) against water and then freeze dried. Characterization data of polymers A and B is presented in Table 3.

TABLE 3
Characterization of polymer A and B.

| | Conversion/% ^{a} | | | | Units^{c} | |
|---|---|---|---|---|---|---|
| Polymers | NIPAM | DMAEMA | Mn (TD)^{b} (kDa) | Ð | NIPAM | DMAEMA |
| A | 70 | 100 | 398.3 | 3.08 | 1764 | 1262 |
| B | 90 | 100 | 529.3 | 3.17 | 2430 | 1619 |

^{a}conversion from ¹H NMR.
^{b}TD: triple detection.
^{c}estimated repeating units of the two monomers from conversion of NMR and Mn of GPC.

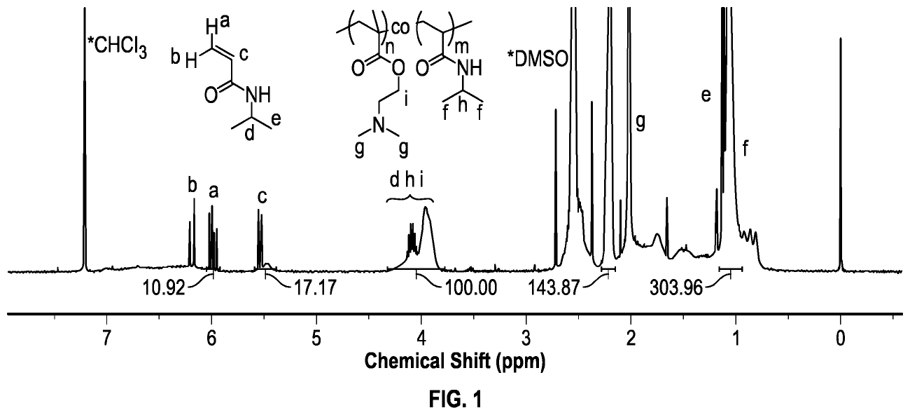

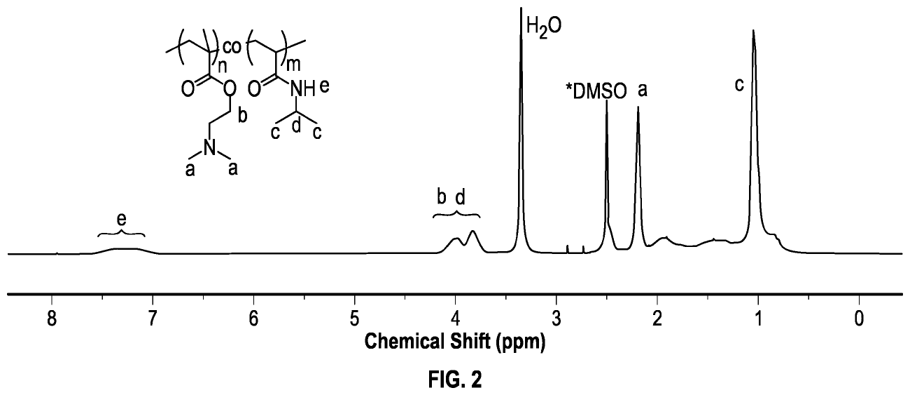

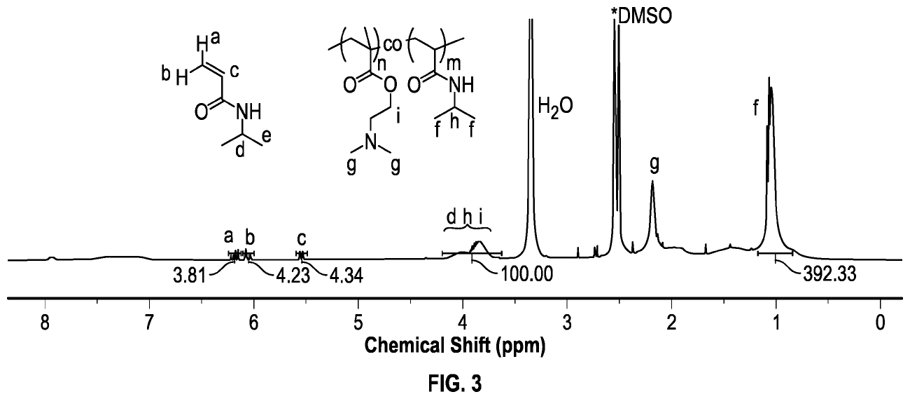

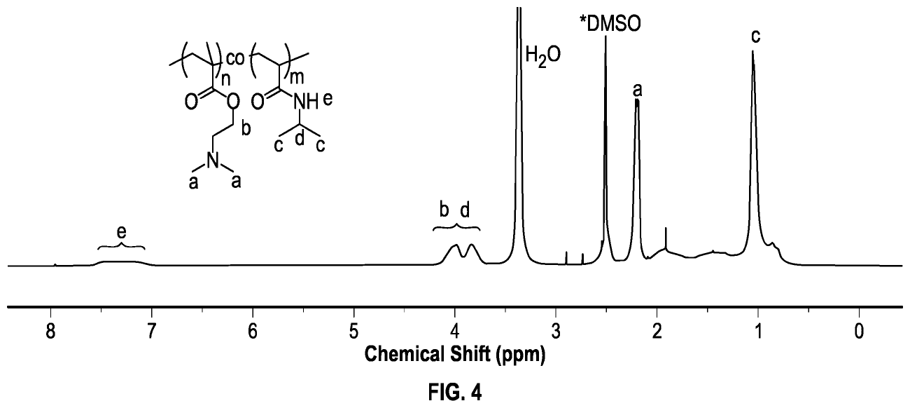

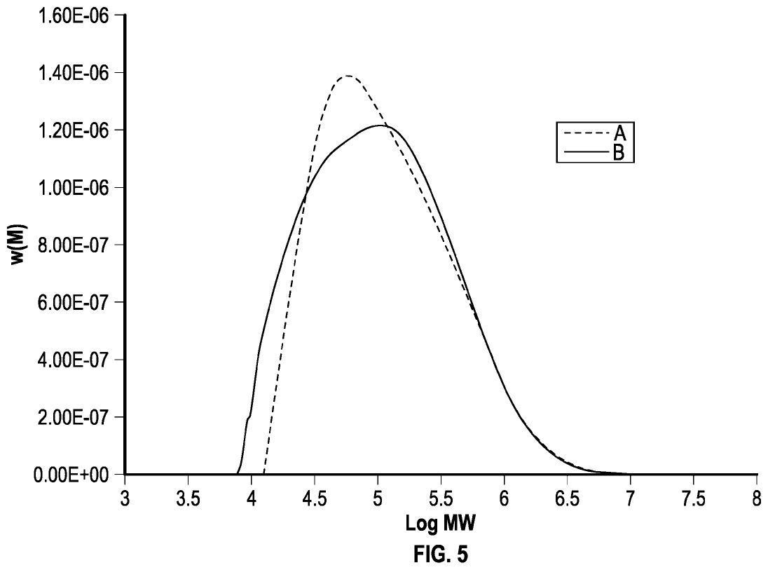

Crude samples and purified samples of polymer A were analyzed using ¹H NMR, as shown in FIGS. 1 and 2, respectively. Crude samples and purified samples of polymer B were analyzed using ¹H NMR, as shown in FIGS. 3 and 4, respectively. Purified samples of polymer A and polymer B were analyzed for molecular weight analysis by SEC, as shown in FIG. 5.

### Polymer Synthetic Procedure

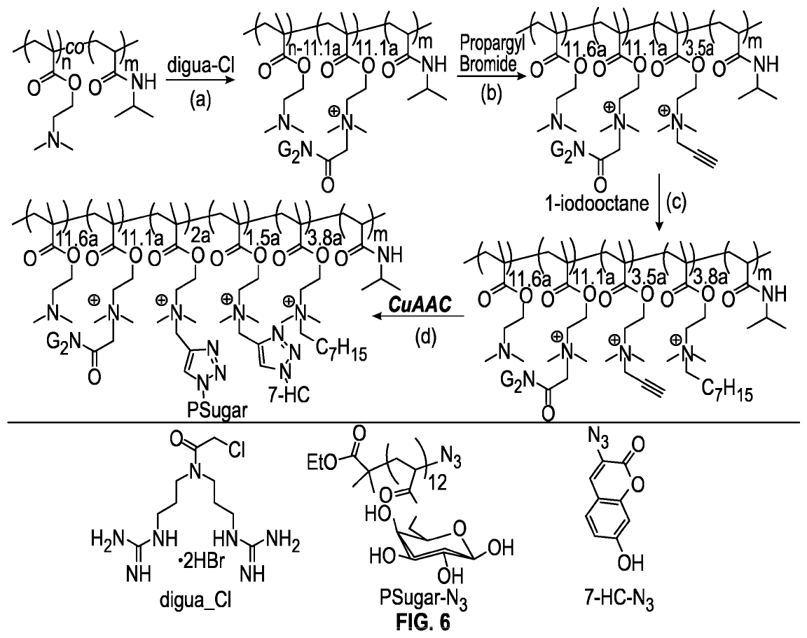

The polymer P(DMAEMA-co-NIPAM) may be functionalized with diguanidine (a), propargyl bromide (b) and iodooctane (c) via quaternization reactions on the DMAEMA amine pendants followed by CuAAC reaction (d) to attach poly(galactose acrylate) azide and 7-hydroxycoumarin azide fluorescent probe, as shown in FIG. 6.

Quaternization of Polymer with Diguanidine Chloroacetamide at 60° C.

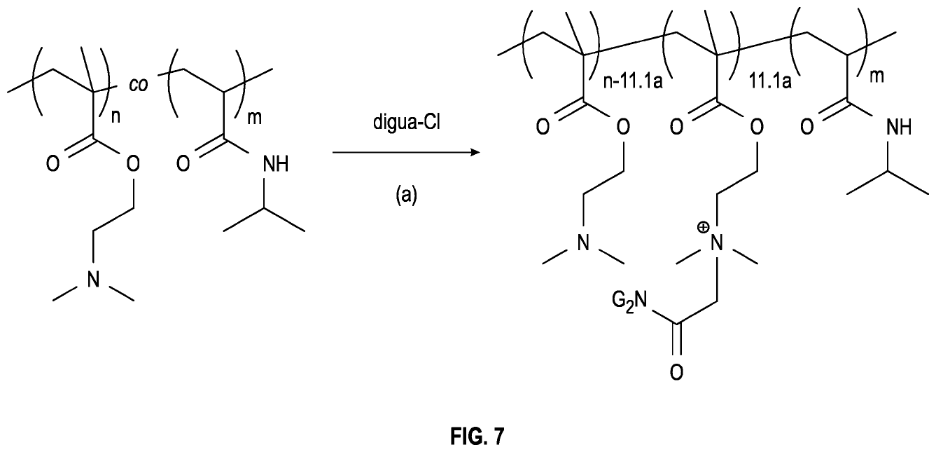

As shown in FIG. 7, polymer A (315 mg, 1 mmol DMAEMA units) was dissolved in ethanol (20 mL) and to this solution was added digua-Cl solution (167.6 mg in 1 mL H₂O) and NaI (55.5 mg) solid. The reaction was then heated for 20 hours at 60° C.

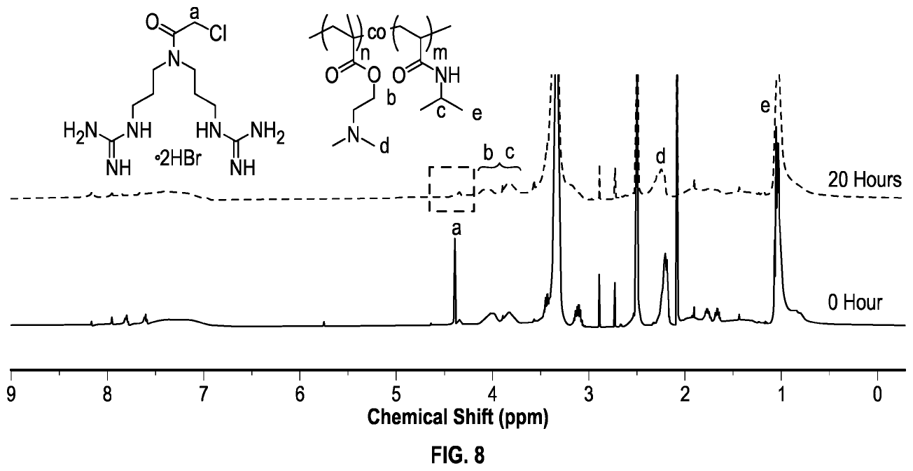

As shown in FIG. 8, samples were taken before and after reaction to determine the conversion of digua-Cl. ¹H NMR shows complete conversion of digua-Cl after 20 hours.

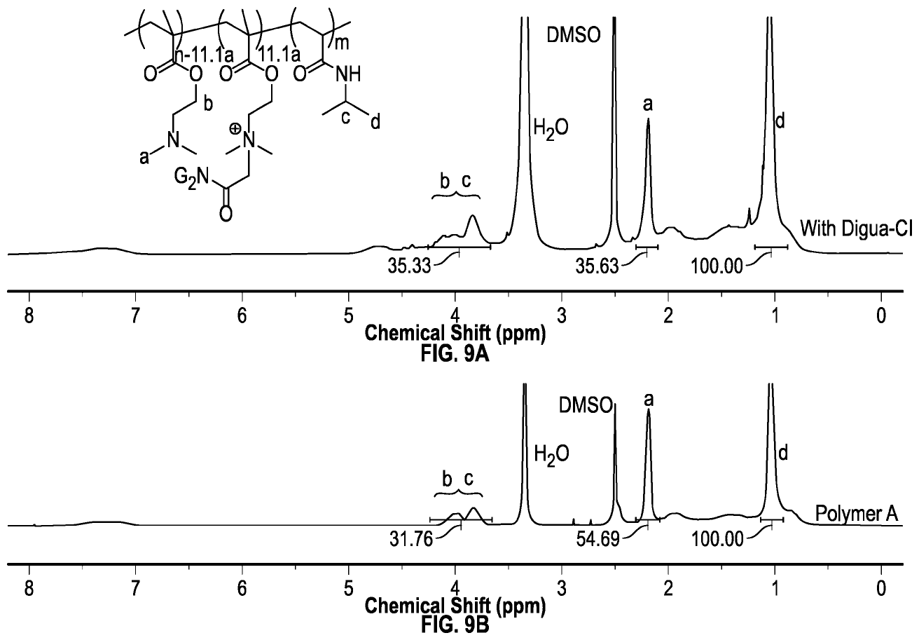

As shown in FIG. 9A, samples were taken and analyzed after the reaction was purified via dialysis (MWCO 10 kDa, against water, 5 buffer changes in 24 h). Samples were compared to the unfunctionalized polymer A, as shown in FIG. 9B. ¹H NMR of the freeze-dried sample showed 35% of the DMAEMA pendants were functionalized with diguanidine. The coupling ratio was (1−35.6/54.7)×100%=35%.

Quaternization of Polymer with Propargyl Bromide

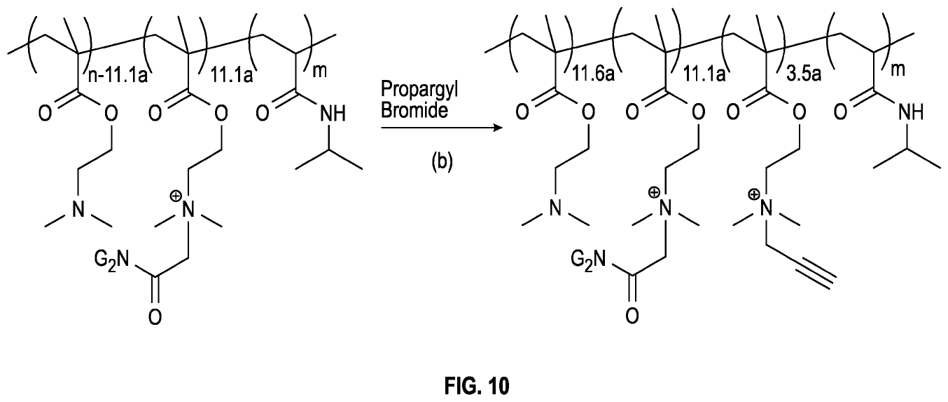

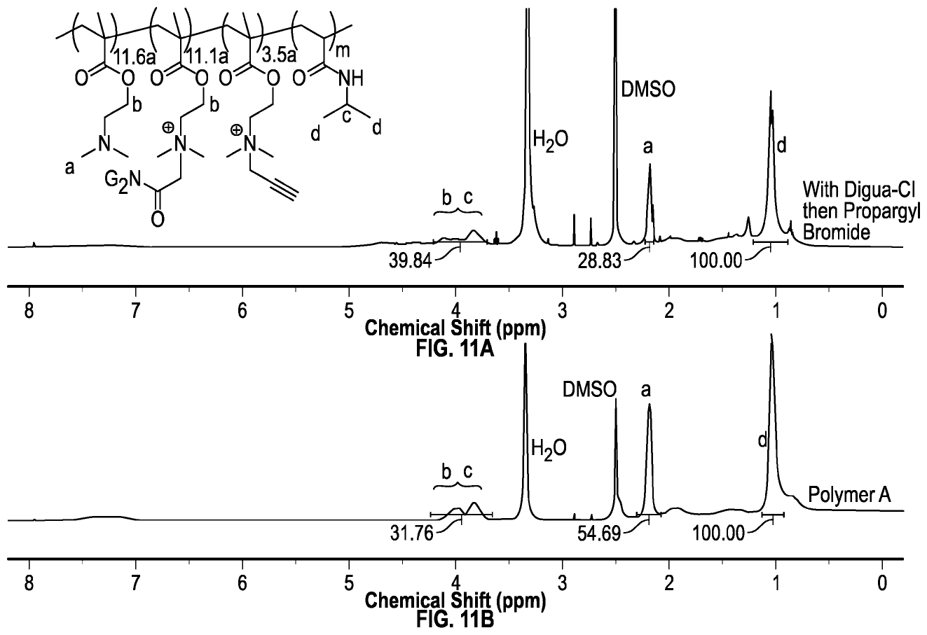

Now referring to FIG. 10, to the solution above was added propargyl bromide (80 wt % in toluene, 13.9 mg, 0.117 mmol) and the reaction was heated at 60° C. for 8 hours before a sample was taken for purification (dialysis against water, MWCO 10 kDa, 5 buffer changes, 24 hours). ¹H NMR of the freeze-dried sample shows 11.7% of the DMAEMA pendants were functionalized with propargyl bromide, as shown in FIG. 11A, which was compared to the unfunctionalized polymer A, as shown in FIG. 11B. The coupling ratio=(35.6−28.8)/54.7×100%=12.4%.

Quaternization of Polymer with 1-Iodooctane

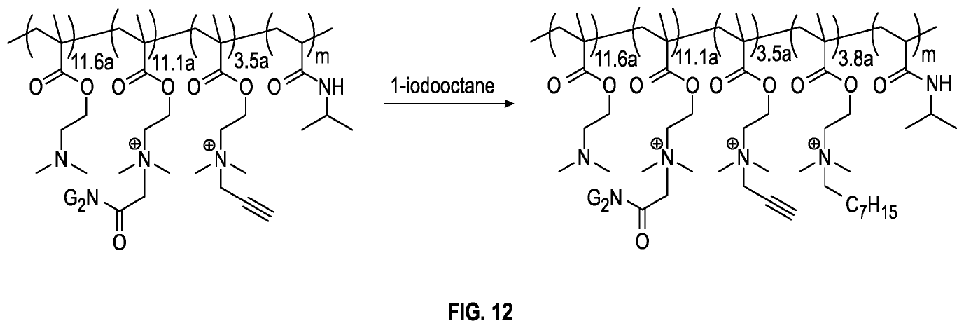

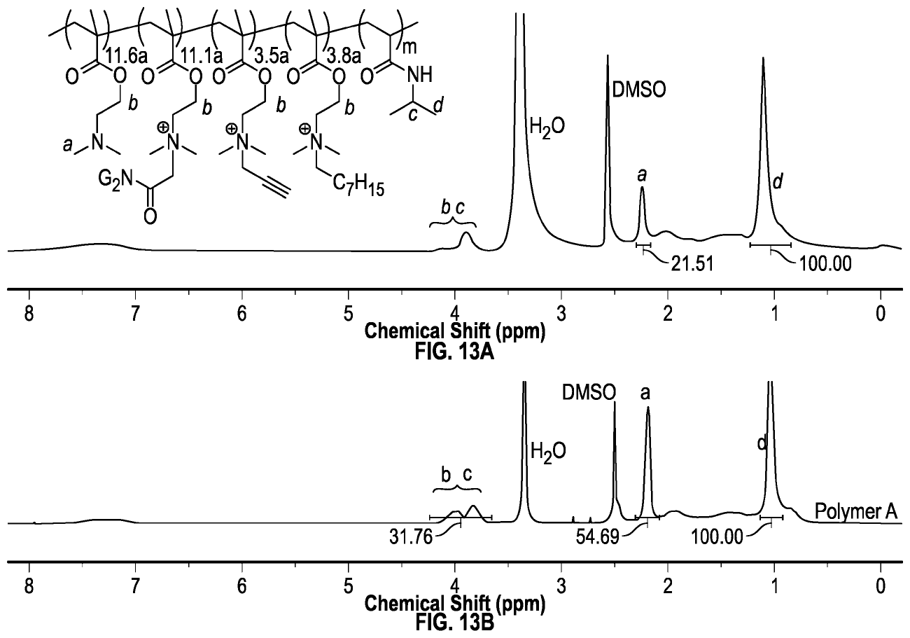

Now referring to FIG. 12, to the solution above was added 1-iodooctane (30.4 mg, 0.127 mmol) and the reaction was heated at 60° C. for 16 hours before a sample was taken for purification (dialysis against water, MWCO 10 kDa, 5 buffer changes, 24 hours). ¹H NMR of the freeze-dried sample shows 13.3% of the DMAEMA pendants were functionalized with iodooctane, as shown in FIG. 13A, which was compared to the unfunctionalized polymer A, as shown in FIG. 13B. The coupling ratio=(28.8−21.5)/54.7×100%=13.3%.

CuAAC Coupling of Polymer with Polysugar Azide and Coumarin Azide.

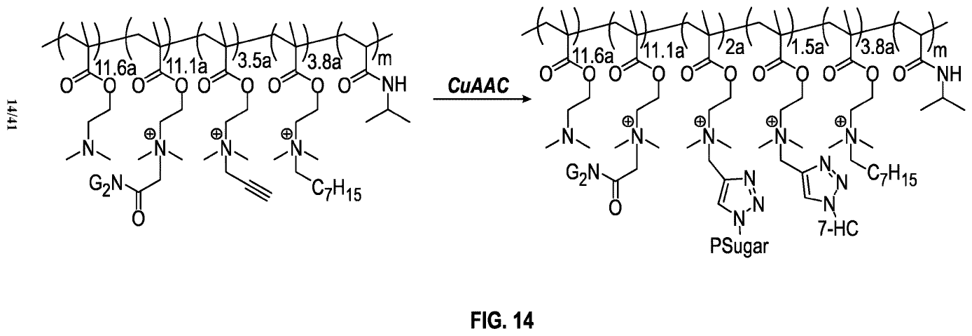

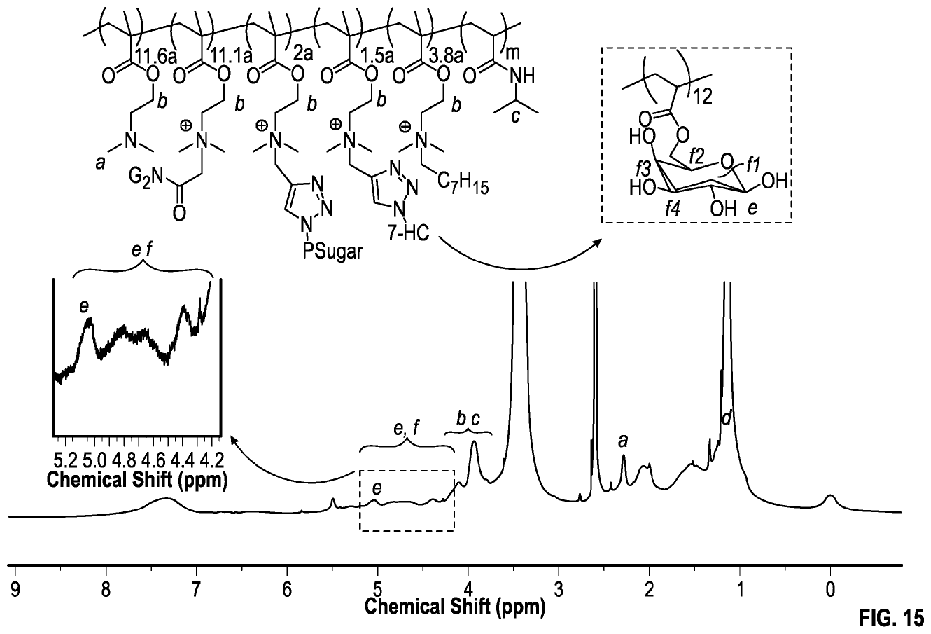

Now referring to FIG. 14, to the solution above (2 mL, 1/10 scale) was added polysugar azide (23.3 mg), azidocoumarin (1.02 mg) and ascorbic acid (13 mg), followed by degassing with argon bubbling for 15 minutes. Then a degassed CuSO₄ solution (18.3 mg in 0.5 mL H₂O) was added via syringe. The reaction was performed at room temperature for 24 hours before addition of EDTA tetrasodium solution (30 mg in 1 mL H₂O). Then the mixture was purified via dialysis (MWCO 10 kDa, against water, 5 buffer changes in 24 h). The sample was not dissolved, forming cloudy mixture. As shown in FIG. 15, purified samples were analysed using ¹H NMR (in DMSO-d6).

### Alternate Polymer Synthetic Procedure

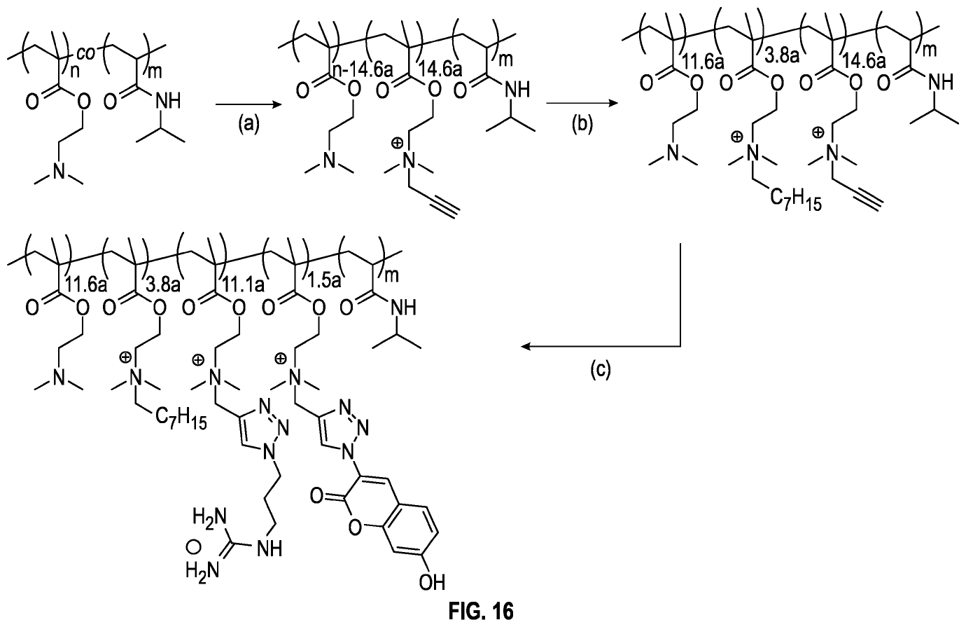

As shown in FIG. 16, the polymer P(DMAEMA-co-NIPAM) was functionalized with propargyl bromide (a) and iodooctane (b) via quaternization reactions on the DMAEMA amine pendants followed by CuAAC reaction (c) to attach poly(galactose acrylate) azide and 7-hydroxycoumarin azide fluorescent probe.

Quaternization of Alternate Polymer with Propargyl Bromide

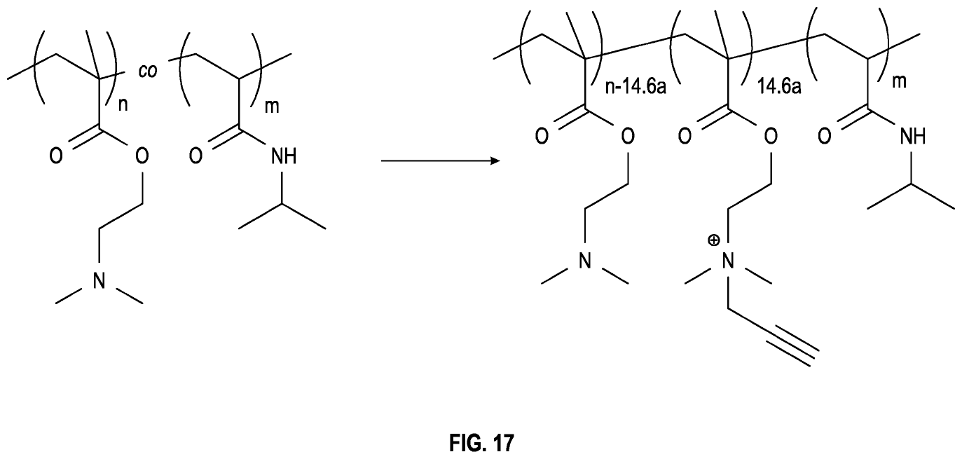

Now referring to FIG. 17, polymer A (328 mg, 1 mmol DMAEMA units) was dissolved in ethanol (20 mL) and to this solution was added propargyl solution (80 wt % in toluene, 72 mg) at room temperature. The reaction was then heated for 8 hours at 60° C.

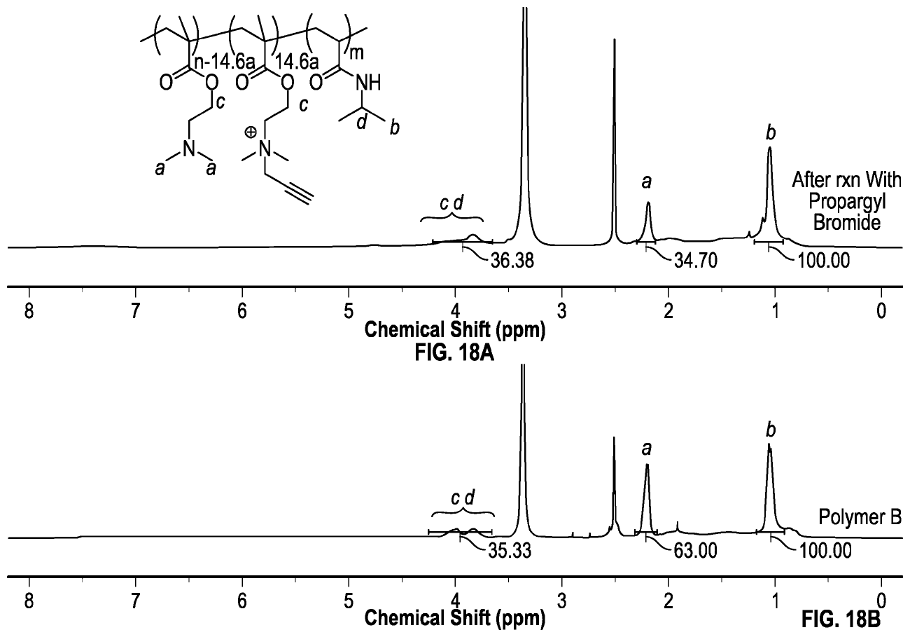

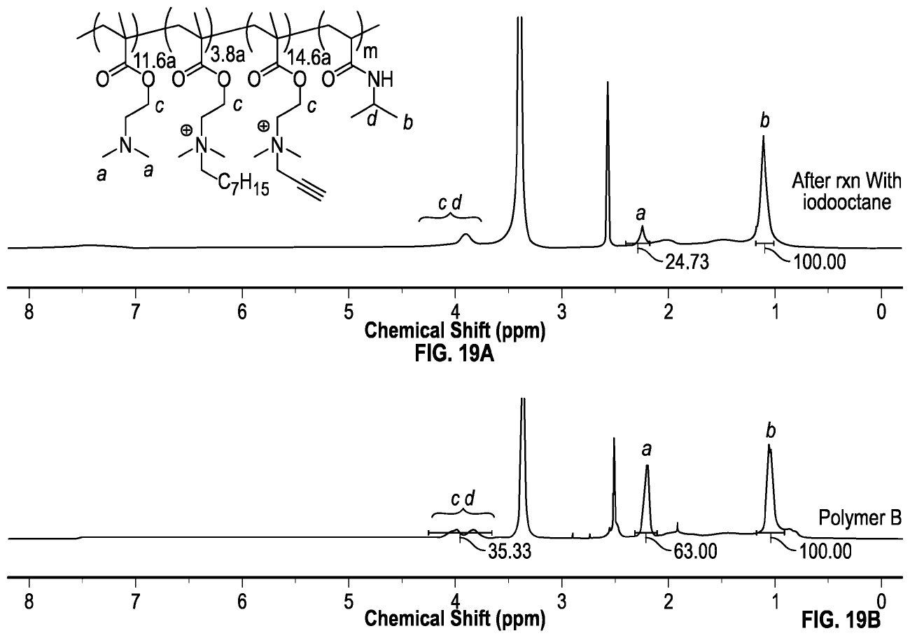

A sample was taken for purification via dialysis (MWCO 10 kDa, against water, 5 buffer changes in 24 hours). ¹H NMR showed % of DMAEMA amine pendants was functionalized were functionalized with propargyl bromide in the purified sample, as shown in FIG. 18A, which was compared to the unfunctionalized polymer B, as shown in FIG. 18B. The coupling ratio=(1−34.7/63)×100%=44.9%.

Quaternization of Alternate Polymer with 1-Iodooctane

To the solution obtained above was added 1-iodooctane (30.4 mg, 0.127 mmol) and the reaction was heated at 60° C. for 16 hours before a sample was taken for purification (dialysis against water, MWCO 10 kDa, 5 buffer changes, 24 hours). ¹H NMR of the freeze-dried sample shows 15.9% of the DMAEMA pendants were functionalized with iodooctane in the purified sample, as shown in FIG. 19A, which was compared to the unfunctionalized polymer B, as shown in FIG. 19B. The coupling ratio=(34.7−24.7/63)×100%=15.9%.

CuAAC Coupling of Alternate Polymer with Polysugar Azide and Coumarin Azide.

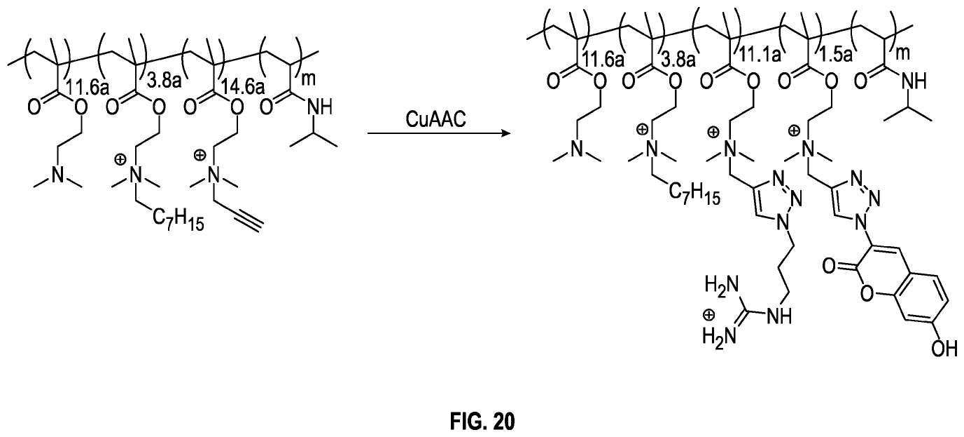

Now referring to FIG. 20, to the solution above (2 mL, 1/10 scale) was added azidopropylguanidine solution (20 wt % in EtOH, 41 mg solution), polysugar azide (23.3 mg), azidocoumarin (1.02 mg) and ascorbic acid (26 mg), followed by degassing with argon bubbling for 15 minutes. Then a degassed CuSO₄ solution (36.5 mg in 0.5 mL H₂O) was added via syringe. The reaction was performed at room temperature for 24 hours before addition of EDTA tetrasodium solution (15 mg in 1 mL H₂O). Then the mixture was purified via dialysis (MWCO 10 kDa, against water, 5 buffer changes in 24 h). The sample was not dissolved during dialysis, forming cloudy mixture.

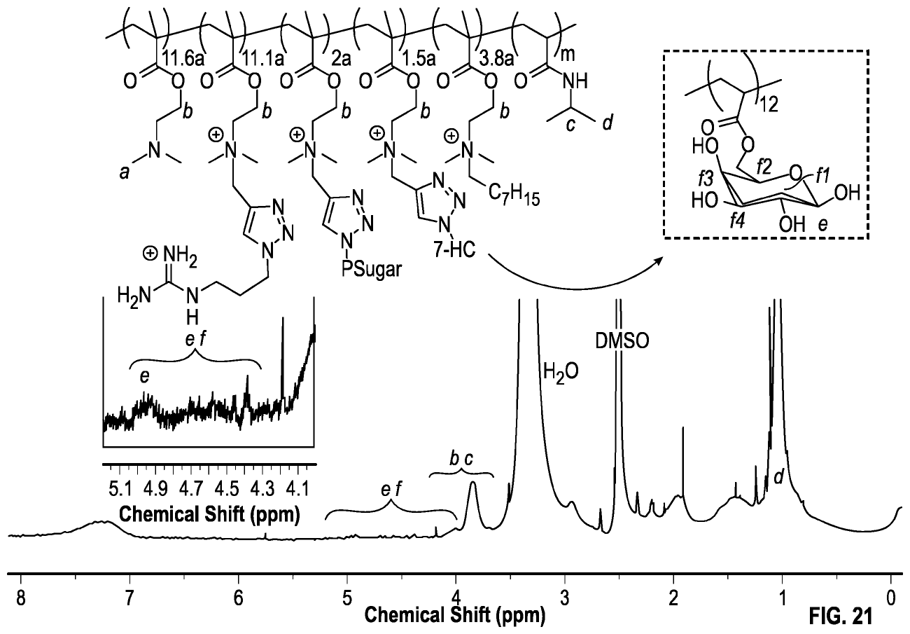

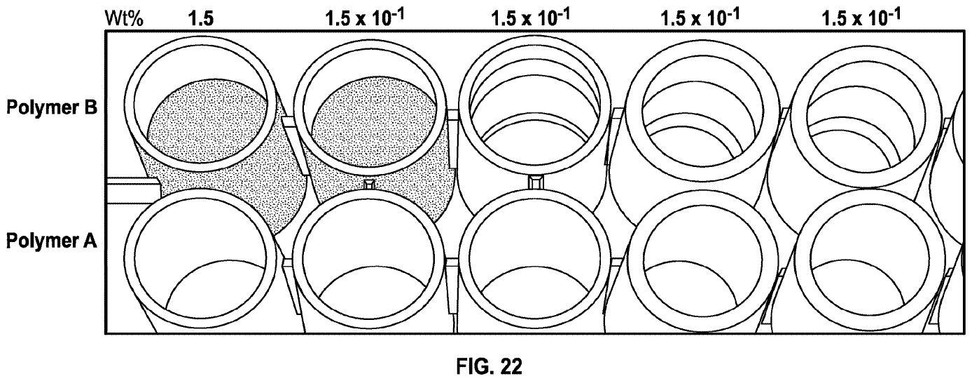

The sample after purification exhibited poor solubility in either DMSO-d6 or D₂O, resulting in poor NMR resolution, as shown in FIG. 21. However, the fluorescence test supports the successful coupling of coumarin via CuAAC, as shown in Table 4 described below.

### Fluorescence Test

The purified samples of the polymer and the alternate polymer were suspended in water to make 1.5 wt % suspension (in 4° C. fridge for 24 h, then 16 hours shaking). Then the two suspensions were diluted 10 to 10000 times, as shown in Table 4 and FIG. 22.

TABLE 4
Fluorescent test of the polymer and the alternate polymer.

| | Fluorescent response for wt % | | | | | |
|---|---|---|---|---|---|---|
| | | 1.5 × | 1.5 × | 1.5 × | 1.5 × | Empty |
| Samples | 1.5 | 10⁻¹ | 10⁻² | 10⁻³ | 10⁻⁴ | plate |
| Alternate Polymer | 935 | 144 | 13 | 4 | 4 | 3 |
| Polymer | 1754 | 588 | 58 | 9 | 4 | 2 |

### Polymer Synthesis Based on RAFT Polymerization

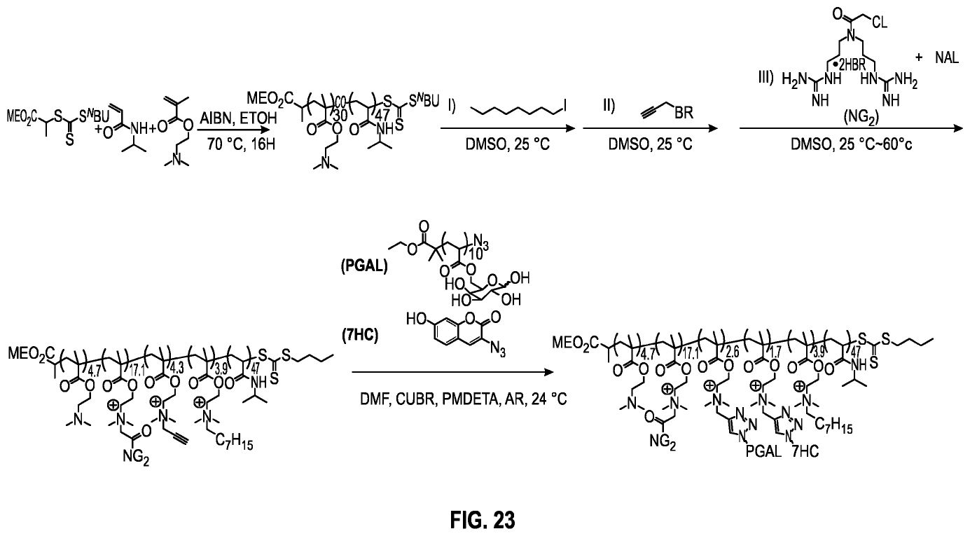

As shown in FIG. 23, the polymer was functionalized with iodooctane (i), propargyl bromide (ii) and diguanidine (iii) via quaternization reactions on the DMAEMA amine pendants followed by CuAAC coupling to attach poly(galactose acrylate) azide and 7-hydroxycoumarin azide.

### Synthesis of MacroCTA-B: P(NIPAM47-co-DMAEMA30)

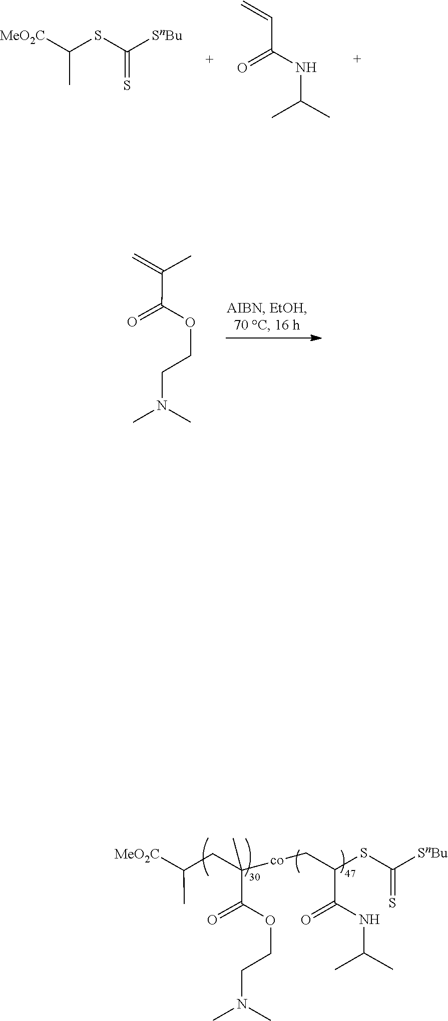

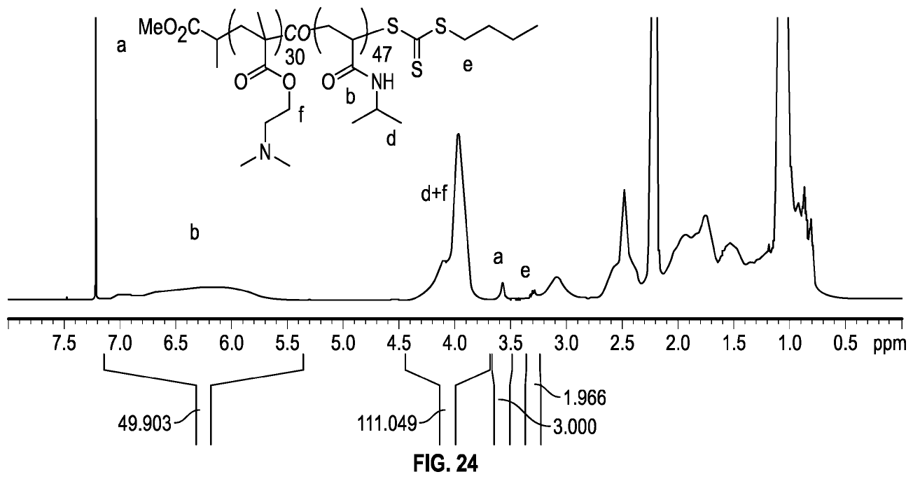

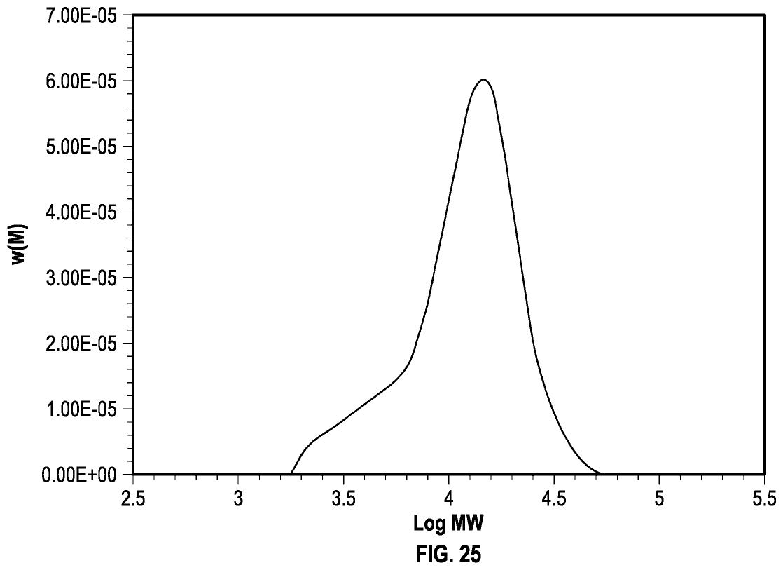

A clean and dried round-bottom flask was used to add AIBN (0.0367 g, 2.23×10⁻⁴ mol) and a RAFT agent (0.3735 g, 1.48×10⁻³ mol). The crude commercial NIPAM (8.4522 g, 7.47×10⁻² mol) and DMAEMA (7.0809 g, 4.5×10⁻² mol) were dissolved in EtOH (18 mL), and 0.05 g of basic Al₂O₃ was added to bind the inhibitor. The mixture was stirred for 2 h, and the resulting solution was filtered through cotton and collected in the round flask. The solution was then degassed with argon gas for approximately 1 h. The polymerization mixture was placed into an oil bath preheated to 70° C. and allowed to polymerize for 16 h. A sample was taken to determine the conversion using ¹H NMR, as shown in FIG. 24. The obtained polymer solution was purified by dialysis (MWCO 3.5 kDa, against tap water, 24 h, 5 buffer changes) for molecular weight analysis by SEC, as shown in FIG. 25. Characterization data is presented below in Table 5.

TABLE 5
Molecular weigh

| | Conv. (%)^{a} | | Mn | SEC | | | Repeating Units^{c} | |
|---|---|---|---|---|---|---|---|---|
| MacroCTA | NIPAM | DMAEMA | Theory^{b} | Mn_{RI} | Mn_{TD} | Ð_{RI} | NIPAM | DMAEMA |
| MacroCTA-B | 94 | 100 | 10287 | 13160 | 10280 | 1.29 | 47 | 30 |

### Quaterization of MacroCTA-B: P(NIPAM47-co-DMAEMA30)

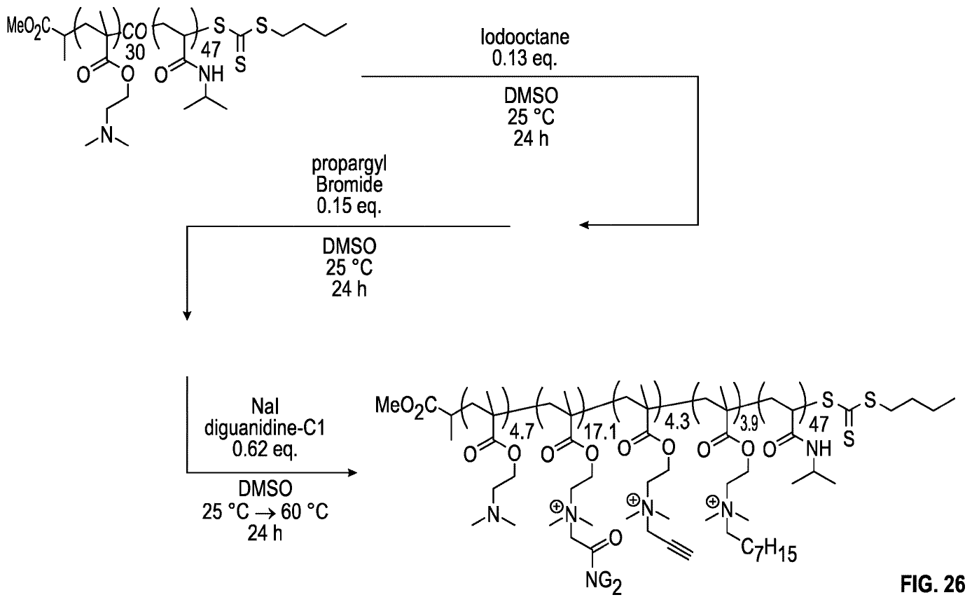

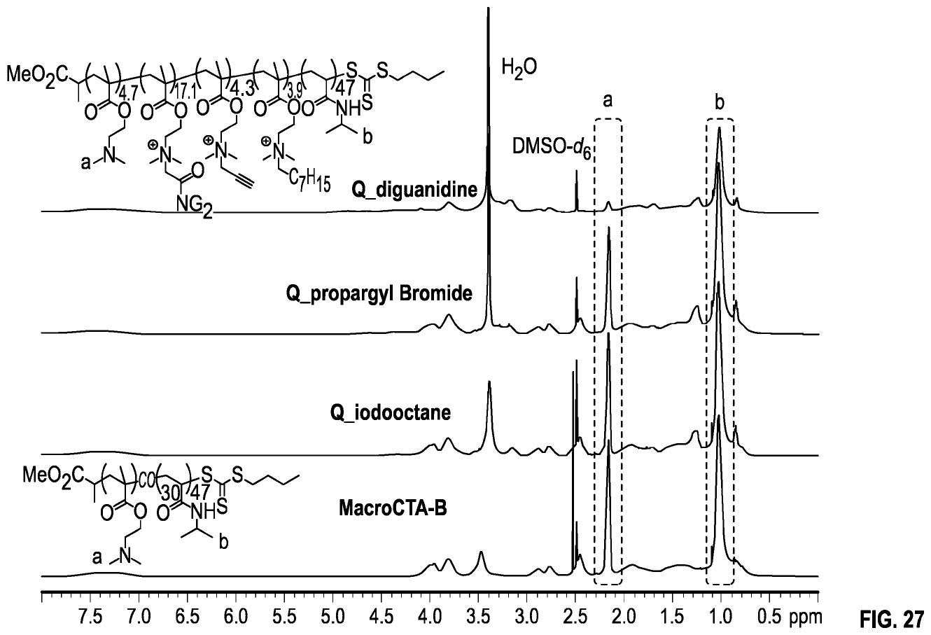

As shown in FIG. 26, MacroCTA-B: P(NIPAM₄₇-co-DMAEMA₃₀) (3.0 g, 8.75×10⁻³ mol DMAEMA group) was dissolved in 10 mL of DMSO and the resulting mixture was placed in a temperature-controlled water bath at 25° C. Iodooctane (0.273 g, 1.14×10⁻³ mol, 0.13 eq. to DMAEMA) was added to the solution, and the reaction was stirred for 24 h. A 2 mL sample of the reaction solution was taken out and purified by dialysis against water for 24 h, then freeze-dried for characterization by ¹H NMR, as shown in FIG. 27.

Propargyl bromide (0.125 g, 1.05×10⁻³ mol, 0.15 eq. to DMAEMA) was added to the remaining solution (8 mL), and the reaction was stirred for 24 h. Following the same procedure as before, a 2 mL sample of the reaction solution was taken out, purified by dialysis against water for 24 h, and freeze-dried for characterization by ¹H NMR, as shown in FIG. 27.

Finally, NaI (0.487 g, 3.25×10⁻³, 0.62 eq. to DMAEMA) and diguanidine-Cl (1.471 g, 3.24×10⁻³, 0.62 eq. to DMAEMA) were added to the remaining solution (6 mL), and the reaction was stirred for 5 min at 25° C. The reaction mixture was then heated to 60° C. and stirred for 24 h. The result solution was purified by dialysis against water for 24 h, and freeze-dried for characterization by ¹H NMR, as shown in FIG. 27.

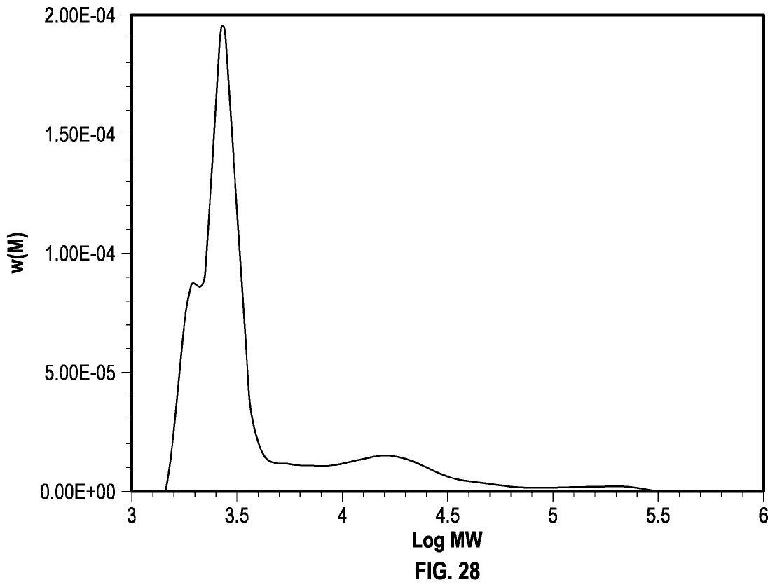

Purified samples of MacroCTA-B were analyzed for molecular weight analysis by SEC, as shown in FIG. 28. Quaternization efficiency was determined by ¹H NMR, shown below in Table 6.

TABLE 6
Quaternization efficiency
Quaternization Efficiency (%)

| Q_iodooctane | Q_propargyl bromide | Q_diguanidine |
|---|---|---|
| 99.93 | 96.09 | 92.4 |

### Functionalization of Quaternized MacroCTA-B by CuAAC

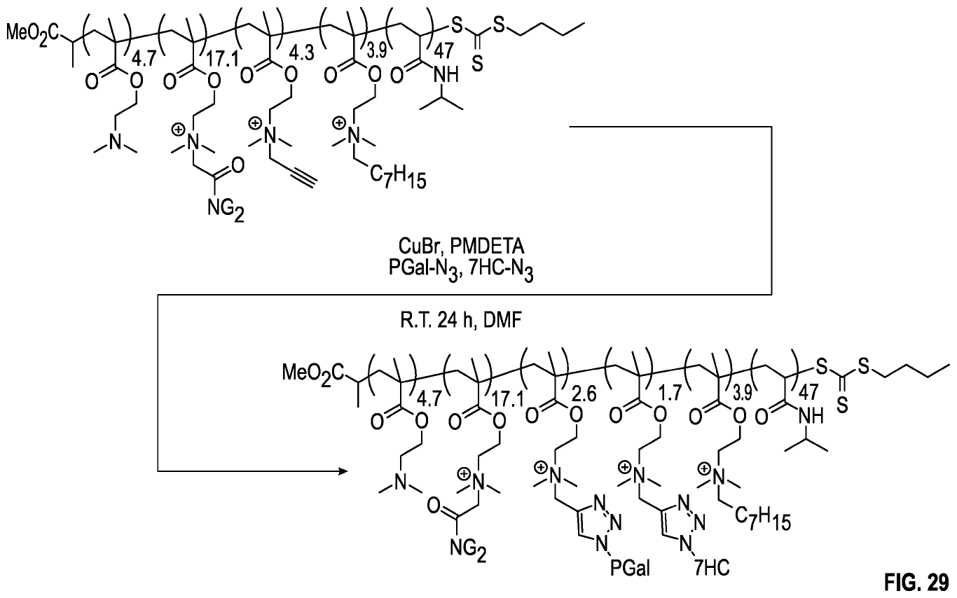

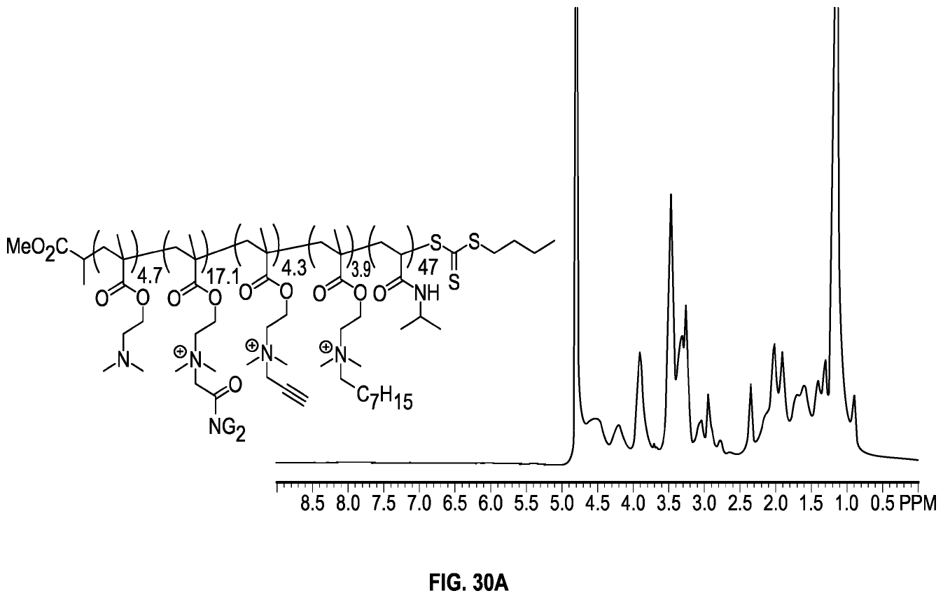

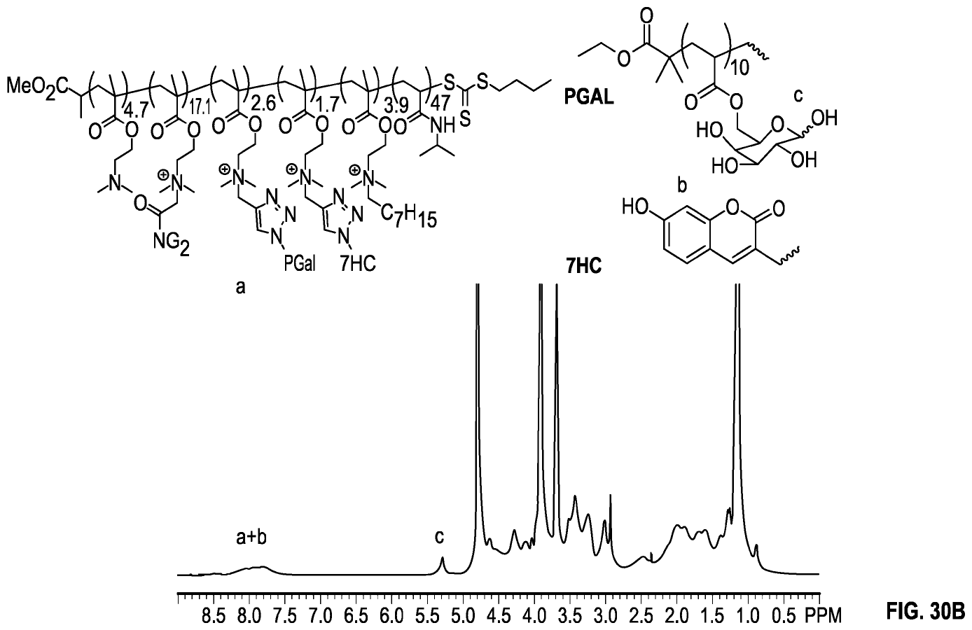

As shown in FIG. 29, CuBr(0.171 g, 1.19×10⁻³ mol, 10 eq. to propargyl group) was placed into a vial and Ar gas was purged into it for 30 min. In another vial, PMDETA (0.250 mL, 1.19×10⁻³ mol, 10 eq. to propargyl group) was dissolved in 5 mL of DMF and purged with Ar for 30 min. P(galactose)-N₃ (0.211 g, 84% N₃ functionalities, 7.14×10⁻⁵ mol-N₃, 0.6 eq. to propargyl group) and 3-azido-7-hydroxycoumarin (0.010 g, 4.76×10⁻⁵ mol, 0.4 eq. to propargyl group) were dissolved in 5 mL of DMF. The solution was also purged with Ar for 30 min. Then, the PMDETA solution in DMF was added to the vial with CuBr to form the CuBr/PMDETA complex. After an additional 5 min of purging, the solution containing P(galactose)-N₃ and 3-azido-7-hydroxycoumarin in DMF was injected into the CuBr/PMDETA solution to initiate the click reaction. The CuAAC reaction was stirred under argon overnight and later purified through dialysis (MWCO 10 kDa) against water for 48 h. The solution was freeze-dried to yield the product, which was then analyzed by ¹H NMR, as shown in FIGS. 30A and 30B.

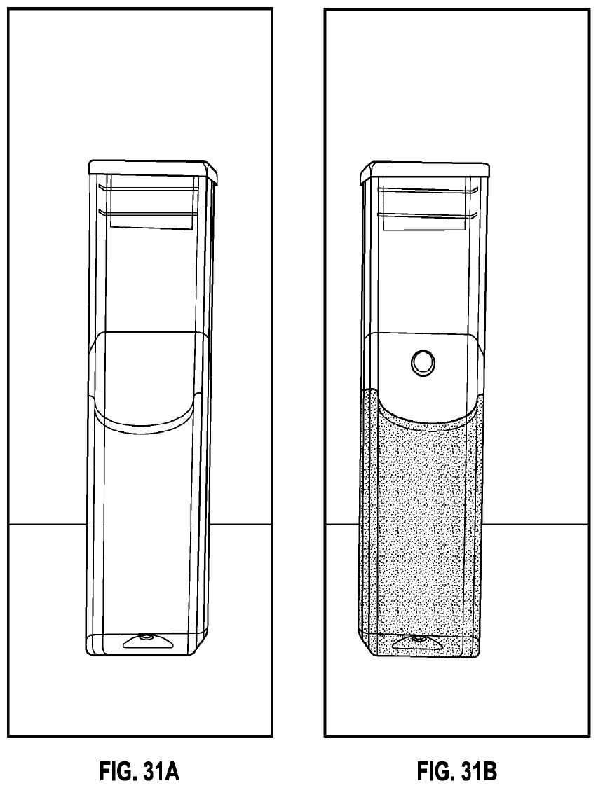

No fluorescence was found prior to coupling of coumarin via CuAAC, as shown in FIG. 31A. However, fluorescence was found after coupling of coumarin via CuAAC, indicating successful coupling, as shown in FIG. 31B.

### Polymer Synthesis Based on Free Radical Polymerization

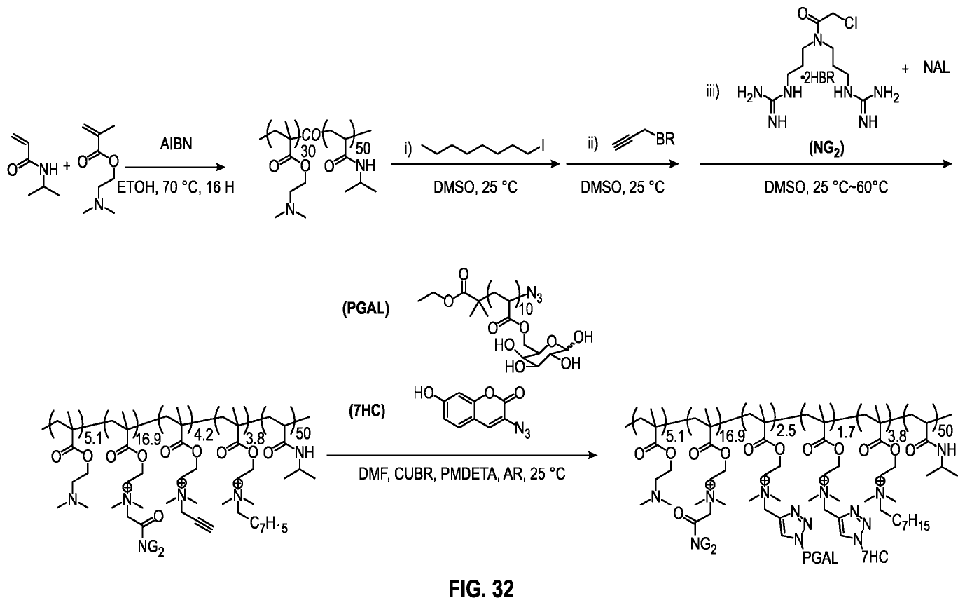

As shown in FIG. 32, the polymer was functionalized using a free radical polymerization processes including iodooctane (i), propargyl bromide (ii) and diguanidine (iii) via quaternization reactions on the DMAEMA amine pendants followed by CuAAC coupling to attach poly(galactose acrylate) azide and 7-hydroxycoumarin azide.

### Free Radical Polymerization of P(NIPAM50-co-DMAEMA30)

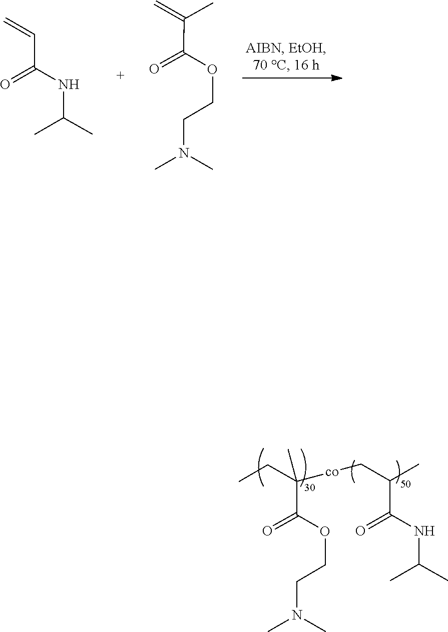

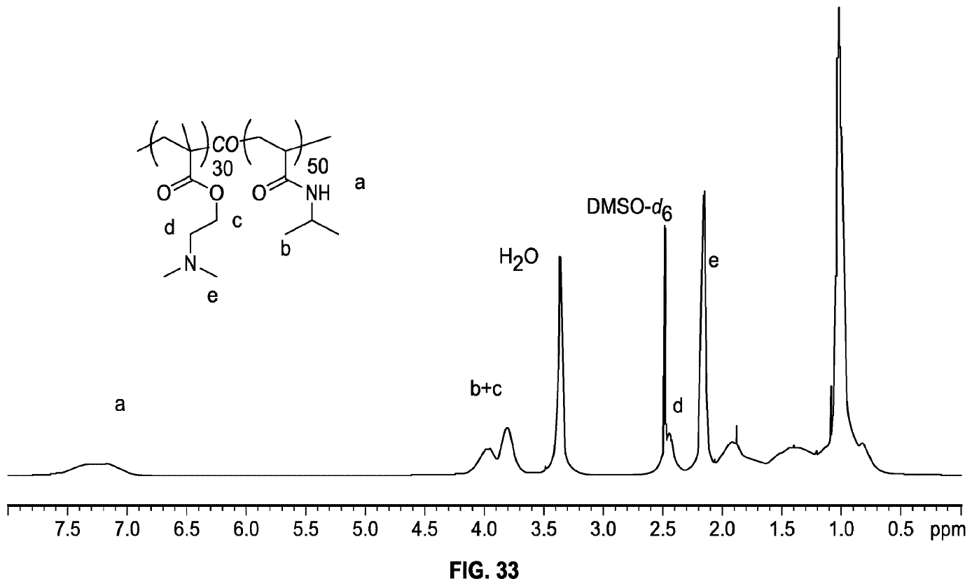

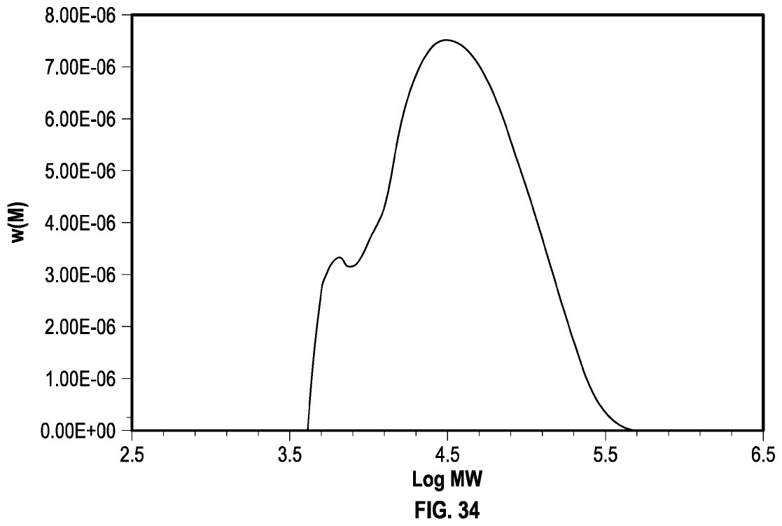

A clean and dried round-bottom flask was used to add AIBN (0.0371 g, 2.23×10⁻⁴ mol). The crude commercial NIPAM (8.4531 g, 7.47×10⁻² mol) and DMAEMA (7.0795 g, 4.5×10⁻² mol) were dissolved in EtOH (18 mL), and 0.05 g of basic Al₂O₃ was added to bind the inhibitor. The mixture was stirred for 2 h, and the resulting solution was filtered through cotton and collected in the round flask. The solution was then degassed with argon gas for approximately 1 hour. The polymerization mixture was placed into an oil bath preheated to 70° C. and allowed to polymerize for 16 h. A sample was taken to determine the conversion using ¹H NMR, as shown in FIG. 33. The obtained polymer solution was purified by dialysis (MWCO 3.5 kDa, against tap water, 24 h, 5 buffer changes) for molecular weight analysis by SEC, as shown in FIG. 34 and Table 7.

TABLE 7
Synthesis of P(NIPAM₅₀-CO-DMAEMA₃₀)

| | Conv. (%)^{a} | | SEC | | | Repeating Units^{b} | |
|---|---|---|---|---|---|---|---|
| Polymer | NIPAM | DMAEMA | Mn_{RI} | Mn_{TD} | Ð_{RI} | NIPAM | DMAEMA |
| P(NIPAM₅₀- | 99 | 100 | 51086 | 10840 | 2.03 | 50 | 30 |
| CO-DMAEMA₃₀) | | | | | | | |

### Quaterization of P(NIPAM50-co-DMAEMA30)

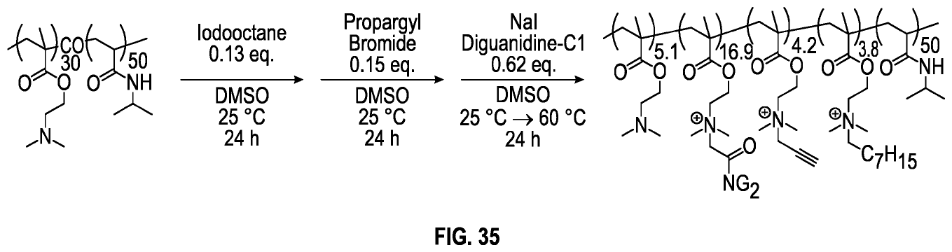

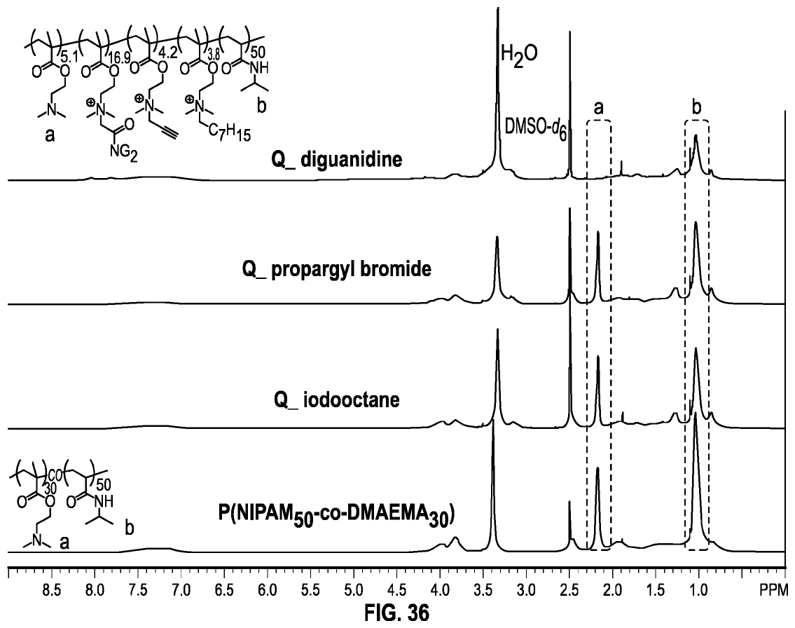

As shown in FIG. 35, P(NIPAM₅₀-co-DMAEMA₃₀) (3.0 g, 8.68×10⁻³ mol DMAEMA group) was dissolved in 30 mL of DMSO and the resulting mixture was placed in a temperature-controlled water bath at 25° C. Iodooctane (0.271 g, 1.13×10⁻³ mol, 0.13 eq. to DMAEMA) was added to the solution, and the reaction was stirred for 24 h. A 6 mL sample of the reaction solution was taken out and purified by dialysis against water for 24 h, then freeze-dried for characterization using ¹H NMR, as shown in FIG. 36.

Propargyl bromide (0.124 g, 1.04×10⁻³ mol, 0.15 eq. to DMAEMA) was added to the remaining solution (24 mL), and the reaction was stirred for 24 h. Following the same procedure as before, a 6 mL sample of the reaction solution was taken out, purified by dialysis against water for 24 h, and freeze-dried for characterization by ¹H NMR, as shown in FIG. 36.

Finally, NaI (0.481 g, 3.21×10⁻³, 0.62 eq. to DMAEMA) and diguanidine-Cl (1.456 g, 3.21×10⁻³, 0.62 eq. to DMAEMA) were added to the remaining solution (18 mL), and the reaction was stirred for 5 min at 25° C. The reaction mixture was then heated to 60° C. and stirred for 24 h. The resulting solution was purified by dialysis against water for 24 h, and freeze-dried for characterization by ¹H NMR, as shown in FIG. 36.

Quaternization efficiency was determined by ¹H NMR, shown below in Table 8.

TABLE 8
Quaternization efficiency
Quaternization Efficiency (%)

| Q_iodooctane | Q_propargyl bromide | Q_diguanidine |
|---|---|---|
| 97.23 | 94.39 | 91.55 |

### Functionalization of Quaternized-P(NIPAM50-co-DMAEMA30) by CuAAC

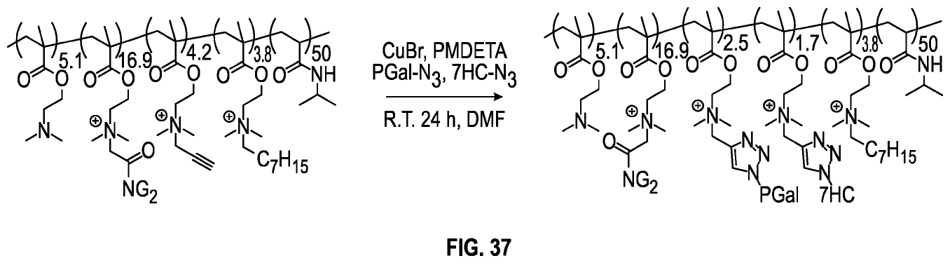

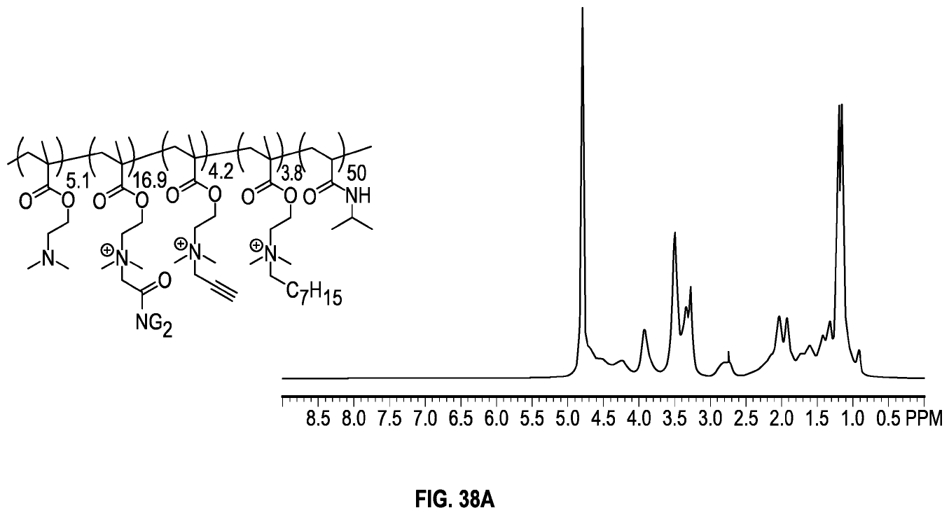

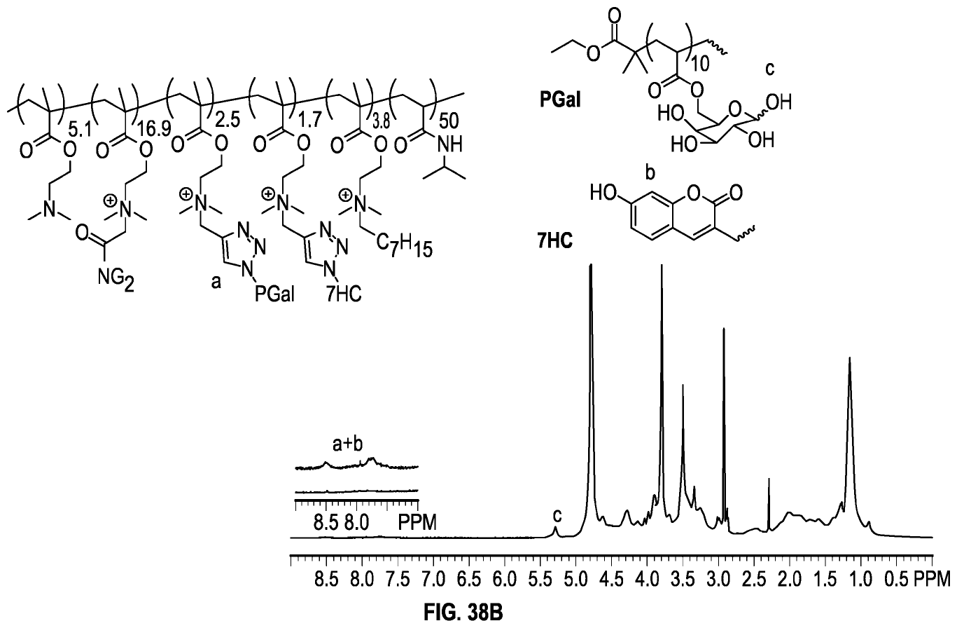

As shown in FIG. 37, CuBr(0.173 g, 1.20×10⁻³ mol, 10 eq. to propargyl group) was placed into a vial and Ar gas was purged into it for 30 min. In another vial, PMDETA (0.250 mL, 1.19×10⁻³ mol, 10 eq. to propargyl group) was dissolved in 15 mL of DMF and purged with Ar for 30 min. P(galactose)-N3 (0.214 g, 84% N₃ functionalities, 7.15×10⁻⁵ mol N₃, 0.6 eq. to propargyl group) and 3-azido-7-hydroxycoumarin (0.011 g, 4.78×10⁻⁵ mol, 0.4 eq. to propargyl group) were dissolved in 15 mL of DMF. The solution was also purged with Ar for 30 min. Then, the PMDETA solution in DMF was added to the vial with CuBr to form the CuBr/PMDETA complex. After an additional 5 min of purging, the solution containing P(galactose)-N₃ and 3-azido-7-hydroxycoumarin in DMF was injected into the CuBr/PMDETA solution to initiate the click reaction. The CuAAC reaction was stirred under argon for the entire night and later purified through dialysis (MWCO 10 kDa) against water for 48 h. The solution was freeze-dried to yield the product, which was then analyzed by ¹H NMR, as shown in FIGS. 38A and 38B.

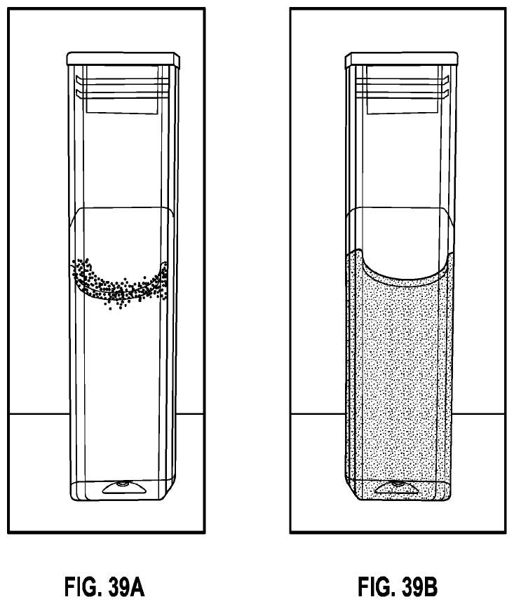

No fluorescence was found prior to coupling of coumarin via CuAAC, as shown in FIG. 39A. However, fluorescence was found after coupling of coumarin via CuAAC, indicating successful coupling, as shown in FIG. 39B.

### ADDITIONAL ASPECTS

Clause 1. A method of disposing a polymer onto a surface, comprising:
depositing the polymer on the surface, the polymer comprising:
a plurality of N-isopropylacrylamide units; a moiety represented by the formula:

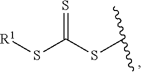

wherein R¹ is C₁-C₂₀ alkyl;
a moiety represented by the formula:

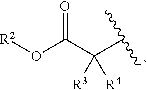

wherein R², R³, and R⁴ are each independently hydrogen or C₁-C₂₀ alkyl;
a plurality of moieties represented by the formula:

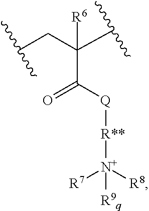

wherein Q is O or N, R** is C₁-C₂₀ alkyl, e.g., methyl, ethyl, propyl, butyl, pentyl, hexyl, heptyl, octyl, nonyl, decyl, or the like, R⁶, R⁷, and R⁸ are independently C₁-C₆ alkyl or hydrogen and R⁹, if present, is C₁-C₁₆ alkyl, C₁-C₆ alkylyne, azole, guanidine, an oligomer of guanidine, such as diguanidine, polysaccharide, chromophore, and combination(s) thereof, q is an integer of 0 or 1, and wherein at least one q is 1.
Clause 2. The method of clause 1, wherein depositing is performed using an aqueous solution comprising the polymer at a concentration of about 0.5 wt % to about 3 wt %.
Clause 3. The method of clause 1, wherein depositing is performed using an aqueous emulsion comprising the polymer at a concentration of about 0.5 wt % to about 3 wt %.
Clause 4. The method of any of clauses 1-3, further comprising evaporating water of the aqueous solution after depositing the aqueous solution onto the surface.
Clause 5. The method of any of clauses 1-4, wherein depositing the structure on the surface is performed by painting the surface, dipping the surface, spraying the surface, taping the surface, brush coating the surface, spin coating the surface, roll coating the surface, doctor-blade coating the surface, or combination(s) thereof with the polymer.
Clause 6. The method of any of clauses 1-5, wherein the surface is a surface of an item of personal protective equipment.
Clause 7. The method of any of clauses 1-6, wherein the surface is an interior or exterior surface of an aircraft, a ship, a train, a boat, a terminal, or a spacecraft.
Clause 8. The method of any of clauses 1-6, wherein the surface is a surface of an air filter of a vehicle.
Clause 9. The method of any of clauses 1-8, wherein the surface is a floor surface, a seat surface, a tray table surface, an overhead bin surface, a ceiling surface, a door surface or a door handle surface.
Clause 10. The method of any of clauses 1-9, further comprising depositing the polymer on a surface comprising a BA.1 SARS-CoV-2 virus.
Clause 11. A polymer comprising:
a plurality of N-isopropylacrylamide units; a moiety represented by the formula:

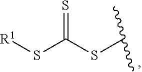

wherein R¹ is C₁-C₂₀ alkyl;
a moiety represented by the formula:

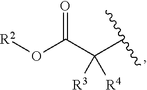

wherein R², R³, and R⁴ are each independently hydrogen or C₁-C₂₀ alkyl;
a plurality of moieties represented by the formula:

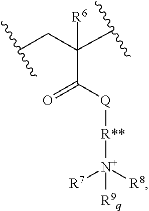

wherein Q is O or N, R** is C₁-C₂₀ alkyl, e.g., methyl, ethyl, propyl, butyl, pentyl, hexyl, heptyl, octyl, nonyl, decyl, or the like, R⁶, R⁷, and R⁸ are independently C₁-C₆ alkyl or hydrogen and R⁹, if present, is C₁-C₁₆ alkyl, C₁-C₆ alkylyne, azole, diguanidine, polysaccharide, chromophore, and combination(s) thereof, q is an integer of 0 or 1, and wherein at least one q is 1.
Clause 12. The polymer of clause 11, wherein the polymer is represented by formula (I):

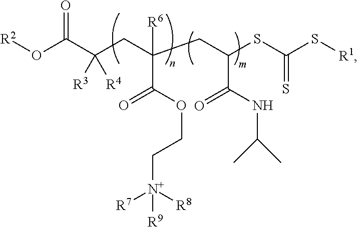

wherein n and m are each independently integers of 1 to 100, q is 0 or 1, and each of R², R³, and R⁴ of Formula (I) are each independently hydrogen or C₁-C₂₀ alkyl, each of R⁶, R⁷, and R⁸ of Formula (I) are C₁-C₆ alkyl or hydrogen, R⁹ of Formula (I), if present, is C₁-C₁₆ alkyl, C₁-C₆ alkylyne, azole, diguanidine, polysaccharide, chromophore, and combination(s) thereof, and wherein each of R¹⁰ and R¹¹ is independently hydrogen or C₁-C₂₀ alkyl.
Clause 13. The polymer of clause 12, wherein n is 20-50.
Clause 14. The polymer of clause 13, wherein n is 30.
Clause 15. The polymer of clause 12, wherein m is 30 to 60.
Clause 16. The polymer of clause 15, wherein m is 45.
Clause 17. The polymer of any of clauses 11-17, wherein R¹ is C₁-C₆ alkyl.
Clause 18. The polymer of clause 17, wherein R¹ is methyl.
Clause 19. The polymer of any of clauses 11-18, wherein R² is C₁-C₆ alkyl.
Clause 20. The polymer of clause 19, wherein R² is methyl.
Clause 21. The polymer of any of clauses 11-20, wherein R³ is C₁-C₆ alkyl.
Clause 22. The polymer of clause 21, wherein R³ is methyl.
Clause 23. The polymer of any of clauses 11-22, wherein R⁹ is C₁-C₁₆ alkyl.
Clause 24. The polymer of clause 23, wherein R⁹ is C₇ alkyl.
Clause 25. The polymer of any of clauses 11-24, wherein R⁹ is C₁-C₆ alkylyne.
Clause 26. The polymer of clause 25, wherein R⁹ is C₃ alkylyne.
Clause 27. The polymer of any of clauses 11-26, wherein R⁹ is azole.
Clause 28. The polymer of clause 27, wherein R⁹ is a guanidine, diguanidine, polygalactose, coumarin, and combination(s) thereof bonded via an azole.
Clause 32. The polymer of any of clauses 11-31, wherein the polymer is represented by formula (II):

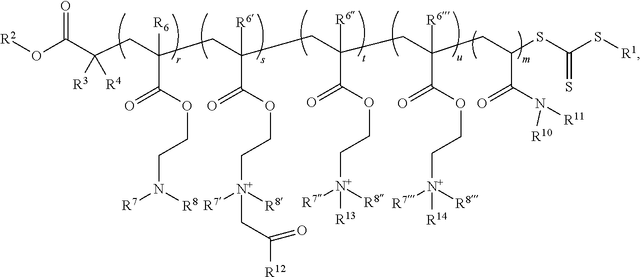

wherein r, s, t, u, and m are integers ranging from 1 to 100, wherein R¹ is butyl, each of R², R³, R⁶, R^{6′}, R^{6″}, R^{6′″}, R⁷, R^{7′}, R^{7″}, R^{7′″}, R⁸, R^{8′}, R^{8″}, and R^{8′″} is methyl, R⁴ is hydrogen, R⁹ is isopropyl, R¹⁰ is hydrogen, R¹¹ is guanidine or diguanidine, R¹² is azole, and R¹³ is octyl.

1. A method of disposing a polymer onto a surface, comprising:
depositing the polymer on the surface, the polymer comprising:
a plurality of N-isopropylacrylamide units; a moiety represented by the formula: 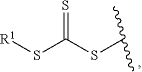
wherein R¹ is C₁-C₂₀ alkyl;
a moiety represented by the formula: 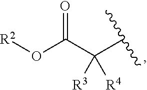
wherein R², R³, and R⁴ are each independently hydrogen or C₁-C₂₀ alkyl;
a plurality of moieties represented by the formula: 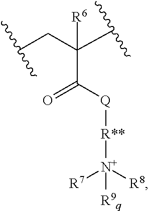
wherein Q is O or N, R** is C₁-C₂₀ alkyl, R⁶, R⁷, and R⁸ are independently C₁-C₆ alkyl or hydrogen and R⁹, if present, is C₁-C₁₆ alkyl, C₁-C₆ alkylyne, azole, guanidine, an oligomer of guanidine such as diguanidine, polysaccharide, chromophore, and combination(s) thereof, q is an integer of 0 or 1, and wherein at least one q is 1.

2. The method of claim 1, wherein depositing is performed using an aqueous solution comprising the polymer at a concentration of about 0.5 wt % to about 3 wt %.

3. The method of claim 1, wherein depositing is performed using an aqueous emulsion comprising the polymer at a concentration of about 0.5 wt % to about 3 wt %.

4. The method of claim 1, further comprising evaporating water of the aqueous solution after depositing the aqueous solution onto the surface.

5. The method of claim 1, wherein depositing the structure on the surface is performed by painting the surface, dipping the surface, spraying the surface, taping the surface, brush coating the surface, spin coating the surface, roll coating the surface, doctor-blade coating the surface, or combination(s) thereof with the polymer.

6. The method of claim 1, wherein the surface is a surface of an item of personal protective equipment.

7. The method of claim 1, wherein the surface is an interior or exterior surface of an aircraft, a ship, a train, a boat, a terminal, or a spacecraft.

8. The method of claim 1, wherein the surface is a surface of an air filter of a vehicle.

9. The method of claim 1, wherein the surface is a floor surface, a seat surface, a tray table surface, an overhead bin surface, a ceiling surface, a door surface, or a door handle surface.

10. The method of claim 1, further comprising depositing the polymer on a surface comprising a BA.1 SARS-CoV-2 virus.

11. A polymer comprising:
a plurality of N-isopropylacrylamide units; a moiety represented by the formula: 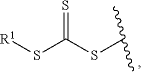
wherein R¹ is C₁-C₂₀ alkyl;
a moiety represented by the formula: 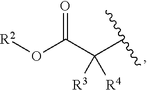
wherein R², R³, and R⁴ are each independently hydrogen or C₁-C₂₀ alkyl;
a plurality of moieties represented by the formula: 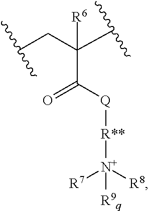
wherein Q is O or N, R** is C₁-C₂₀ alkyl, R⁶, R⁷, and R⁸ are independently C₁-C₆ alkyl or hydrogen and R⁹, if present, is C₁-C₁₆ alkyl, C₁-C₆ alkylyne, azole, guanidine, an oligomer of guanidine such as diguanidine, polysaccharide, chromophore, and combination(s) thereof, q is an integer of 0 or 1, and wherein at least one q is 1.

12. The polymer of claim 11, wherein the polymer is represented by formula (I): 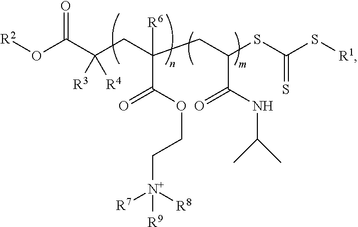
wherein n and m are each independently integers of 1 to 100, q is 0 or 1, and each of R², R³, and R⁴ of Formula (I) are each independently hydrogen or C₁-C₂₀ alkyl, each of R⁶, R⁷, and R⁸ of Formula (I) are C₁-C₆ alkyl or hydrogen, R⁹ of Formula (I), if present, is C₁-C₁₆ alkyl, C₁-C₆ alkylyne, azole, diguanidine, polysaccharide, chromophore, and combination(s) thereof, and wherein each of R¹⁰ and R¹¹ is independently hydrogen or C₁-C₂₀ alkyl.

13. The polymer of claim 12, wherein n is 20-50.

14. The polymer of claim 12, wherein m is 30-60.

15. The polymer of claim 11, wherein each of R¹, R², R³, R⁴, R⁶, R⁷, and R⁸ are independently C1-C6 alkyl.

16. The polymer of claim 11, wherein each of R¹, R², R³, R⁶, R⁷, and R⁸ are independently methyl.

17. The polymer of claim 11, wherein R⁹ is C₇ alkyl.

18. The polymer of claim 11, wherein R⁹ is C₃ alkylyne

19. The polymer of claim 11, wherein the polymer is represented by formula (II): 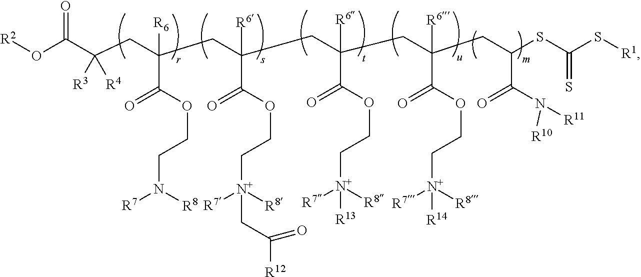
wherein r, s, t, u, and m are integers ranging from 1 to 100, wherein R¹ is butyl, each of R², R³, R⁶, R^{6′}, R^{6″}, R^{6′″}, R⁷, R^{7′}, R^{7″}, R^{7′″}, R⁸, R^{8′}, R^{8″}, and R^{8′″} is methyl, R⁴ is hydrogen, R⁹ is isopropyl, R¹⁰ is hydrogen, R¹¹ is guanidine, an oligomer of guanidine such as diguanidine, R¹² is azole, and R¹³ is octyl.

20. The polymer of claim 19, wherein R¹¹ is diguanidine.
